(19) United States
(12) Patent Application Publication
(10) Pub. No.: US 2012/0148633 A1
(43) Pub. Date: Jun. 14, 2012

(54) BINARY AND TERTIARY GALVANIC PARTICULATES AND METHODS OF MANUFACTURING AND USE THEREOF

(76) Inventors: Ying Sun, Belle Mead, NJ (US); Jue-Chen Liu, Belle Mead, NJ (US); Michael Southall, Lawrenceville, NJ (US); Luiz Arthur Bonaci Tessarotto, Plainsboro, NJ (US); Leon B. Kriksunov, Ithaca, NY (US)

(21) Appl. No.: 13/260,416

(22) PCT Filed: Mar. 25, 2010
(86) PCT No.: PCT/US10/28695

§ 371 Date: Oct. 26, 2011

Related U.S. Application Data
(60) Provisional application No. 61/164,198, filed on Mar. 27, 2009.

(51) Int. Cl. A61K 9/14; A61K 33/38; A61K 33/34; A61K 33/26; A61K 33/32; A61P 17/00; A61K 33/06; A61P 29/00; A61P 31/00; A61P 17/02; A61P 17/10; A61K 33/24; A61K 33/30

(52) U.S. Cl. 424/400; 424/649; 424/618; 424/630; 424/646; 424/639; 424/641; 424/682

(57) ABSTRACT

The present invention relates to galvanic particulates, their methods of manufacture and uses in treatments are described. The galvanic particulates may be binary or tertiary galvanic particulates, for example, containing multiple layers or phases of conductive materials.

The present invention relates to galvanic particulates, their methods of manufacture and uses in treatments are described. The galvanic particulates may be binary or tertiary galvanic particulates, for example, containing multiple layers or phases of conductive materials.

### BACKGROUND OF THE INVENTION

Using a galvanic couple as the power source in iontophoresis patch devices is well known in the art, but less known as a power source for electric stimulation. A typical galvanic couple is made from a pair of dissimilar metals, such as a zinc donor electrode and a silver/silver chloride counter electrode in some galvanic couple powered skin patch devices. These devices are often applied to the human body in order to provide an intended benefit, such as electric stimulation, improving wound healing or enhancing percutaneous drug penetration. Another type of topical system powered by a galvanic couple in the form of particulates is disclosed in U.S. Pat. Nos. 7,476,221, 7,479,133, 7,477,939, 7,476,222, and 7,477,940, U.S. Patent Application Nos. 2005/0148996 and US2007/0060862, and PCT Publication No. 2009/045720 for various beneficial effects.

The present invention provides novel forms of galvanic particulates, methods of manufacturing them, and uses of galvanic particulates in a number of new applications, including methods of treating the human body, including topical tissue applications and internal treatments via various routes of administrations such as peroral, injectable and surgical implants.

### SUMMARY OF THE INVENTION

The present invention provides a multiphase galvanic particulate comprising a dispersed phase comprising a second conductive material dispersed in a continuous phase comprising a first conductive material, wherein both said first conductive material and said second conductive material are exposed on the surface of said particulate, the particle size of said particulate is from about 1 to about 500 microns, and said particulate comprises about 0.5 to about 60 weight percent of said dispersed phase.

The invention also provides methods of making the above multiphase galvanic particulate, and uses thereof, particularly uses for topical application of the same.

The present invention also relates to binary and tertiary galvanic particulates comprising a first conductive material and a second conductive material that may also comprise additional conductive materials and/or substrates and/or conductive/resistive interlayers.

The invention also provides methods of making the above binary and tertiary galvanic particulates, and uses thereof, particularly uses for topical application of the same.

### BRIEF DESCRIPTION OF THE FIGURES

FIG. 1a is a perspective view of a binary galvanic particulate 100.

FIG. 1b is a perspective view of a tertiary galvanic particulate 150.

FIG. 2 is a cross-sectional view of a tertiary galvanic particulate 200 comprising a conductive/resistive interlayer.

FIG. 3 is a cross-sectional view of a tertiary galvanic particulate 300 comprising a porous conductive/resistive interlayer.

FIG. 4a is a perspective view of a binary galvanic particulate of cylindrical shape 400 comprising concentrically located first and second conductive materials 401 and 402.

FIG. 4b is a perspective view of a binary galvanic particulate of cylindrical shape 405 comprising concentrically located first and second conductive materials 451 and 452, coated over a non-conducting cylindrical substrate 455.

FIG. 5a is a perspective view of a tertiary galvanic particulate of cylindrical shape 500 comprising concentrically located first and second conductive materials 501 and 502 and an additional conductive material 503 between them.

FIG. 5b is a perspective view of a tertiary galvanic particulate of cylindrical shape 550 comprising concentrically located first and second conductive materials 551 and 552 and an additional conductive material 553 between them, coated over a non-conducting cylindrical substrate 555.

FIG. 6 is a cross-sectional view of a multiphase galvanic particulate 600 comprising a dispersed phase 602 dispersed in a continuous phase 601 and exposed on the surface.

FIG. 7a is a cross-sectional view of a binary galvanic particulate 700 having a two-layer structure.

FIG. 7b is a cross-sectional view of a binary galvanic particulate 750 comprising a layer of first conductive material 751, a substrate 755, and a layer of second conductive material 752.

FIG. 8 is a cross-sectional view of a galvanic particulate 800 with a four-layer structure.

FIG. 9 is a cross-sectional view of a galvanic particulate 900 with a three-layer structure.

FIG. 10a is a cross-sectional view of a galvanic particulate 1000 with a two-layer structure.

FIG. 10b is a cross-sectional view of a galvanic particulate 1050 with a three-layer structure.

FIG. 10c is a cross-sectional view of a galvanic particulate 1070 with a four-layer structure.

FIG. 11 is a cross-sectional view of a combination galvanic particulate 1110 with a four-layer structure.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

### DEFINITIONS

What is meant by a “product” is a product containing the galvanic particulates (or a composition containing the galvanic particulates) in finished packaged form. In one embodiment, the product contains instructions directing the user ingest, topically apply, or otherwise administer the galvanic particulates or composition (e.g., to treat a skin condition). Such instructions may be printed on the outside of the product, a label insert, or on any additional packaging.

In one aspect, the present invention features promoting the galvanic particulates or a composition containing the galvanic particulates of the present invention for an intended use. What is meant by “promoting” is promoting, advertising, or marketing. Examples of promoting include, but are not limited to, written, visual, or verbal statements made on the product or in stores, magazines, newspaper, radio, television, internet, and the like.

As used herein, “pharmaceutically-acceptable” means that the ingredients which the term describes are suitable for its intended use (e.g., suitable of ingestion or contact with the skin or mucosa) without undue toxicity, incompatibility, instability, irritation, allergic response, and the like.

As used herein, “safe and effective amount” means an amount of the ingredient or the composition sufficient to provide the desired benefit at a desired level, but low enough to avoid serious side effects. The safe and effective amount of the ingredient or composition will vary with the area being treated, the age of the end user, the duration and nature of the treatment, the specific ingredient or composition employed, the particular pharmaceutically-acceptable carrier utilized, and like factors.

As used herein, the term “treating” or “treatment” means the treatment (e.g., alleviation or elimination of symptoms and/or cure) and/or prevention or inhibition of the condition (e.g., a skin, mucosal, or nail condition). In one embodiment, the galvanic particulates are administered locally or systemically to the subject (e.g., a human) in need to such treatment. In one embodiment, the galvanic particulates are used to exert their effects on (i.e., to treat, to improve the health of, to cure, to eliminate and/or to reduce the quantity of) a living organism, including vertebrate animals (mammals including human, birds, fish, reptiles, and amphibian), insects, plants, micro-organisms (e.g., bacteria, fungi and viruses).

### Galvanic Particulates

The galvanic particulates of the present invention include a first conductive material and a second conductive material, wherein both the first conductive material and the second conductive material are exposed on the surface of the particulate. Generally, the first conductive material is a material or metal that is more easily oxidized, and has a more negative value in the Standard Potential Table (e.g., zinc or magnesium), than the second conductive material, which is relatively more difficult to oxidize (or more easily reduced or more noble) and often has a positive Standard Potential value (e.g., copper or silver). Certain materials such as iron have intermediate Standard Potential values and can be used as either the first conductive material or the second conductive material depending on the ease of oxidation (or nobility) of the other conductive material in the galvanic couple.

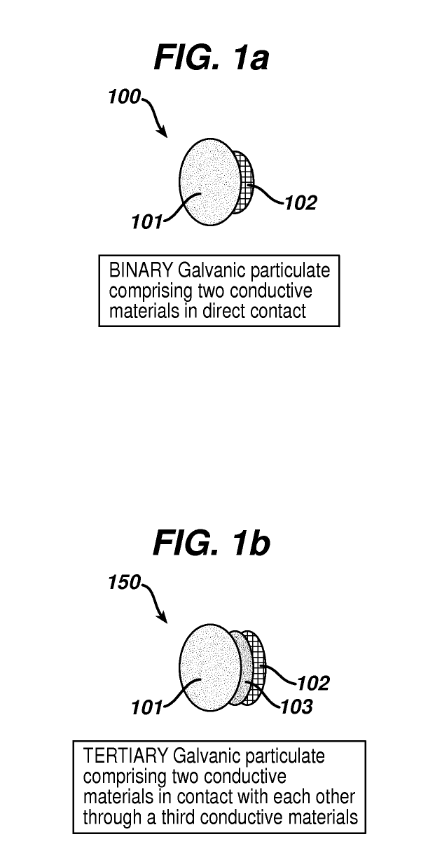

Referring now to FIG. 1a, in one embodiment, the galvanic particulate 100 includes the first conductive material 101, the surface of which is partially coated with the second conductive material 102, for example, the first and second conductive materials are two dissimilar metals in direct contact with each other. Such galvanic particulates are one example of binary galvanic particulates.

In one embodiment, the galvanic particulate comprises one or more additional conductive materials. FIG. 1b depicts layered galvanic particulate comprising first and second conductive materials 101 and 102 in electric contact with each other through an additional conductive material 103. Such a galvanic particulate is referred to hereinafter as a tertiary galvanic particulate. The additional conductive material can be substantially inert or not inert in aqueous environments, while at least one of the first and second conductive materials is substantially not inert in aqueous environments.

The additional conductive material can have an electric conductivity similar to that of the first and/or second conductive materials. Alternatively, the additional conductive material may have a lower electric conductivity than either the first or second conductive material for the purpose of regulating generation of galvanic electricity (e.g., reducing the galvanic current). In this embodiment, the additional conductive material forms a conductive/resistive interlayer between the first and second conductive materials.

The presence of a conductive/resistive interlayer enables control of the discharge current of galvanic particulates when these particles are in contact with electrolytes. Higher resistance of the conductive/resistive interlayer results in slower electrochemical reaction of first and second conductive materials due to higher resistance to the galvanic current between first and second conductive materials and thus in slower overall reaction of the galvanic particulate. Both the nature (conductivity) and the size (thickness) of the conductive/resistive interlayer may be adjusted to control current. Thus, galvanic particulates can be developed having a range of longer and shorter action capabilities. In addition, when using a conductive/resistive interlayer more electrochemically active materials can be selected as first and second conductive materials.

In one embodiment, mixtures of galvanic particulates having different conductive/resistive interlayers are provided in desired proportions. Such a mixture provides long-lasting, steady action. Optionally, a mixture can include both binary and tertiary galvanic particulates.

In one embodiment, the conductive/resistive interlayer material comprises carbon, carbon-based ink, a composite comprising a mixture of non-conductive binder and conductive particles or fillers such as carbon particles, conductive graphite, metal powders, conductive polymer, conductive adhesive, zinc oxide, or other material. The conductive/resistive interlayer can also be a modified form of the first or second conductive material, for example an oxide, halide or other salt, or another compound of the first or second conductive material. The conductive/resistive interlayer can also comprise a conversion coating, for example a phosphate conversion coating developed on the interfacial surface of the first or second conductive material. Other modifications of a surface of the first or second conductive material in the interfacial area between the first and second conductive materials can be used to make the conductive/resistive interlayer.

In one embodiment the conductive/resistive interlayer material comprises a conductive polymer. In one embodiment the conductive/resistive interlayer comprises a composite of a substantially electrically non-conductive polymeric material filled with substantially electrically conductive filler, such as carbon, metallic powder, or similar material.

In one embodiment, the electric conductivity of the conductive/resistive interlayer is higher than conductivity of typical electric insulators such as rubber and lower than conductivity of good electric conductors such as metallic conductors. In another embodiment, the electric conductivity of the conductive/resistive interlayer is below approximately 5×10⁷ S/m, which characterizes conductivity of copper, and above approximately 1×10⁻¹³ S/m which characterizes conductivity of rubber.

In one embodiment, the electric conductivity of the conductive/resistive interlayer is approximately 2.8*10⁴ S/m, which characterizes conductivity of carbon. In another embodiment, the electric conductivity of the conductive/resistive interlayer is in the range of about 1×10⁴ S/m to about 1×10⁶ S/m.

All the above conductivity values are conductivities at ambient temperature.

The thickness of the conductive/resistive interlayer between the first and second conductive materials is from approximately 1 nanometer to approximately 500 microns.

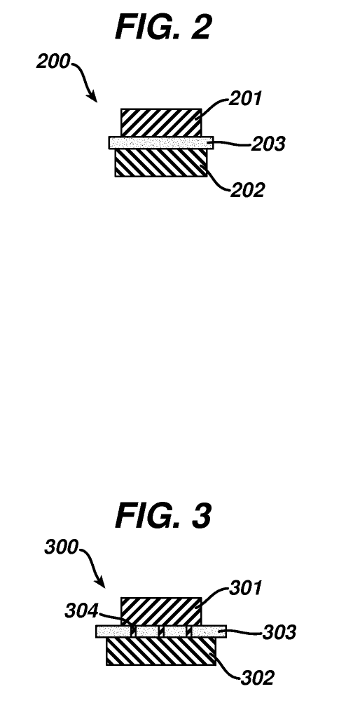

Referring now to FIG. 2, in one embodiment, a conductive/resistive interlayer 203 completely separates a first conductive material 201 and second conductive material 202 in a galvanic particulate 200.

In another embodiment, the conductive/resistive interlayer comprises pores. Referring now to FIG. 3, the first conductive material 301 and second conductive material 302 of a galvanic particulate 300 contact each other through a conductive/resistive interlayer 303 comprising pores 304. The conductive/resistive interlayer can for example be a micro-porous or nano-porous insulating or semi-insulating or semi-conductive material, such as oxide, salt, or other compound of first or second conductive materials, or a polymeric material, for example polyethylene, polystyrene, polypropylene, polyethylene terephthalate, polyester, or a similar polymer. A non-limiting example of such a galvanic particulate is a metallic zinc particle (first conductive metal) with a thin zinc oxide formed on its surface, onto which there is a partial coating of metallic copper. Such zinc-copper particulates with a thin zinc oxide interlayer can be manufactured by physical vapor deposition of copper onto fine metallic zinc powder pretreated with an oxidation process to form a zinc oxide covered surface.

In one embodiment, the galvanic particulates are produced by a coating method wherein the weight percentage of the second conductive material is from about 0.001% to about 20%, by weight, of the total weight of the particulate, such as from about 0.01% to about 10%, by weight, of the total weight of the particulate. In one embodiment, the coating thickness of the second conductive material may vary from single atom up to hundreds of microns. In yet another embodiment, the surface of the galvanic particulate comprises from about 0.001 percent to about 99.99 percent such as from about 0.1 to about 99.9 percent of the second conductive material.

In one embodiment, the galvanic particulates are produced by a non-coating method (e.g., by sintering, melting and dispersing, printing or mechanical processing the first and the second conductive materials together to form the galvanic particulate) wherein the second conductive material comprises from about 0.1% to about 99.9%, by weight, of the total weight of the particulate, such as from about 0.1% to about 90%, or in certain embodiments from about 0.5% to about 60%, preferably from about 0.5% to about 60%, of the total weight of the particulate.

In one embodiment, the galvanic particulates are fine enough that they can be suspended in the semi-solid compositions during storage. In a further embodiment, they are in flattened and/or elongated shapes. The advantages of flattened and elongated shapes of the galvanic particulates include a lower apparent density and, therefore, a better floating/suspending capability in the topical composition, as well as better coverage over the biological tissue, leading to a wider and/or deeper range of the galvanic current passing through the biological tissue (e.g., the skin or mucosa membrane). In one embodiment, the longest dimension of the galvanic particulates is at least twice (e.g., at least five times) the shortest dimension of such particulates.

The galvanic particulates may be of any shape, including but not limited to, spherical or non-spherical particles or elongated or flattened shapes (e.g., cylindrical, fibers or flakes). In one embodiment, the average particle size of the galvanic particulates is from about 10 nanometers to about 500 micrometers, such as from about 100 nanometers to about 100 micrometers. What is meant by the particle size is the maximum dimension in at least one direction.

In one embodiment, the galvanic particulate comprises at least 90 percent, by weight, of conductive materials (e.g., the first conductive material and the second conductive material), such as at least 95 percent, by weight, or at least 99 percent, by weight, when a coating method is used for the production of the galvanic particulates.

In one embodiment, the first conductive material is selected from the group consisting of zinc, magnesium, aluminum, oxides thereof, halides thereof and alloys thereof.

In another embodiment, second conductive material is selected from the group consisting of copper, silver, gold, manganese, iron and alloys thereof.

Examples of combinations of first conductive materials and second conductive materials include (with a “/” sign representing an oxidized but essentially non-soluble form of the metal), but are not limited to, zinc-copper, zinc-copper/copper halide, zinc-copper/copper oxide, magnesium-copper, magnesium-copper/copper halide, zinc-silver, zinc-silver/silver oxide, zinc-silver/silver halide, zinc-silver/silver chloride, zinc-silver/silver bromide, zinc-silver/silver iodide, zinc-silver/silver fluoride, zinc-gold, zinc-carbon, magnesium-gold, magnesium-silver, magnesium-silver/silver oxide, magnesium-silver/silver halide, magnesium-silver/silver chloride, magnesium-silver/silver bromide, magnesium-silver/silver iodide, magnesium-silver/silver fluoride, magnesium-carbon, aluminum-copper, aluminum-gold, aluminum-silver, aluminum-silver/silver oxide, aluminum-silver/silver halide, aluminum-silver/silver chloride, aluminum-silver/silver bromide, aluminum-silver/silver iodide, aluminum-silver/silver fluoride, aluminum-carbon, copper-silver/silver halide, copper-silver/silver chloride, copper-silver/silver bromide, copper-silver/silver iodide, copper-silver/silver fluoride, iron-copper, iron-copper/copper oxide, copper-carbon iron-copper/copper halide, iron-silver, iron-silver/silver oxide, iron-silver/silver halide, iron-silver/silver chloride, iron-silver/silver bromide, iron-silver/silver iodide, iron-silver/silver fluoride, iron-gold, iron-conductive carbon, zinc-conductive carbon, copper-conductive carbon, magnesium-conductive carbon, and aluminum-carbon.

The first conductive material or second conductive material may also be alloys, particularly the first conductive material. Non-limiting examples of the alloys include alloys of zinc, iron, aluminum, magnesium, copper and manganese as the first conductive material and alloys of silver, copper, stainless steel and gold as second conductive material.

In another embodiment, the galvanic particulate can comprise a plurality of conductive materials or metals, namely, the number can be greater than 2 (binary) or 3 (tertiary). A non-limiting example of such a galvanic particulate can have the composition of magnesium-zinc-iron-copper-silver-gold in the form of multiple coatings, multiphase phases, or as a multiple conductive metal composite.

In one embodiment, the galvanic particulate comprises the first conductive material partially coated with several conductive materials, such as with a second and one or more additional conductive materials. In a further embodiment, the particulate comprises at least 95 percent, by weight, of the first conductive material, the second conductive material, and the additional conductive material. In one embodiment, the first conductive material is zinc, the second conductive material is copper, and the additional conductive material is silver.

In one embodiment, the difference of the Standard Electrode Potentials (or simply, Standard Potential) of the first conductive material and the second conductive material is at least about 0.1 volts, such as at least 0.2 volts. In one embodiment, the materials that make up the galvanic couple have a standard potential difference equal to or less than about 3 volts. For example, for a galvanic couple comprised of metallic zinc and copper, the Standard Potential of zinc is −0.763V (Zn/Zn2⁺), and the Standard Potential of copper is +0.337 (Cu/Cu2⁺), the difference of the Standard Potential is therefore 1.100V for the zinc-copper galvanic couple. Similarly, for the magnesium-copper galvanic couple, Standard Potential of magnesium (Mg/Mg2⁺) is −2.363V, and the difference of the Standard Potential is therefore 2.700V. Additional examples of Standard Potential values of some materials suitable for galvanic couples are: Ag/Ag⁺: +0.799V, Ag/AgCl/Cl⁻:0.222V, and Pt/H₂/H⁺:0.000V. Pt may also be replaced by carbon or another conductive material. See, e.g., Physical Chemistry by Gordon M. Barrow, 4^{th} Ed., McGraw-Hill Book Company, 1979, Page 626.

### Manufacture of Galvanic Particulates

In one embodiment, the conductive electrodes are combined (e.g., the second conductive electrode is deposited onto the conductive/resistive interlayer, the first conductive electrode, or a substrate) by chemical, electrochemical, physical or mechanical process (such as electroless deposition, electric plating, gas phase deposition, such as CVD and PVD, vacuum vapor deposition, arc spray, sintering, compacting, pressing, extrusion, printing, and granulation) conductive metal ink (e.g., with polymeric binders), and other known metal coating and powder processing methods commonly used in powder metallurgy, electronics and medical device manufacturing processes, such as the methods described in the book: “ASM Handbook Volume 7: Powder Metal Technologies and Applications” (by ASM International Handbook Committee, edited by Peter W. Lee, 1998, pages 31-109, 311-320); or described in the book Materials and Processes in Manufacturing, E. P. DeGarmo et al., 8^{th} edition, Prentice-Hall, 1997, pages 1096-1110). In another embodiment, all the conductive electrodes are manufactured by chemical reduction processes (e.g., electroless deposition), sequentially or simultaneously, in the presence of reducing agent(s). Examples of reducing agents include phosphorous-containing reducing agents (e.g., a hypophosphite as described in U.S. Pat. Nos. 4,167,416 and 5,304,403), boron-containing reducing agents, and aldehyde- or ketone-containing reducing agents such as sodium tetrahydridoborate (NaBH₄) (e.g., as described in US 20050175649).

In one embodiment, the second conductive electrode is deposited or coated onto the conductive/resistive interlayer present on the first conductive electrode by physical deposition, such as immersion coating, spray coating, plasma coating, conductive ink coating, screen printing, dip coating, metals bonding, bombarding particulates under high pressure-high temperature, fluid bed processing, or vacuum deposition.

In one embodiment, the coating method is based on displacement chemical reaction, namely, contacting a particulate of the first conductive material (e.g., metallic zinc particle) with a solution containing a dissolved salt of the second conductive material (e.g. copper acetate, copper lactate, copper gluconate, or silver nitrate). In a further embodiment, the method includes flowing the solution over the particulate of the first conductive material (e.g., zinc powder) or through the packed powder of the first conductive material. In one embodiment, the salt solution is an aqueous solution. In another embodiment, the solution is contains an organic solvent, such as an alcohol, a glycol, glycerin or other commonly used solvents in pharmaceutical production to regulate the deposition rate of the second conductive material onto the surfaces of the first particulates, therefore controlling the activity of the galvanic particulates produced.

### Coated Wires or Fibers Method of Manufacturing

In one embodiment, galvanic particulates are manufactured by one-layer or multi-layer coating or plating of wires or fibers having diameter from about 10 microns to about 500 microns which are then shredded thus forming galvanic particulates. The length of the shredded particles can vary from less than the diameter of the coated wire to about 20 times the diameter of the coated wire or more. In another embodiment the length of the shredded particles varies from approximately 20 microns to approximately 2 mm. Methods of coating wires or fibers are known in the art. Such methods include, but are not limited to, electroplating, electroless plating, immersion plating, PVD, CVD, plasma deposition, sputtering deposition, or other wire plating or coating technique known in the art, for example by techniques described in U.S. Pat. No. 3,957,452 or 7,220,316.

### Binary Particulates

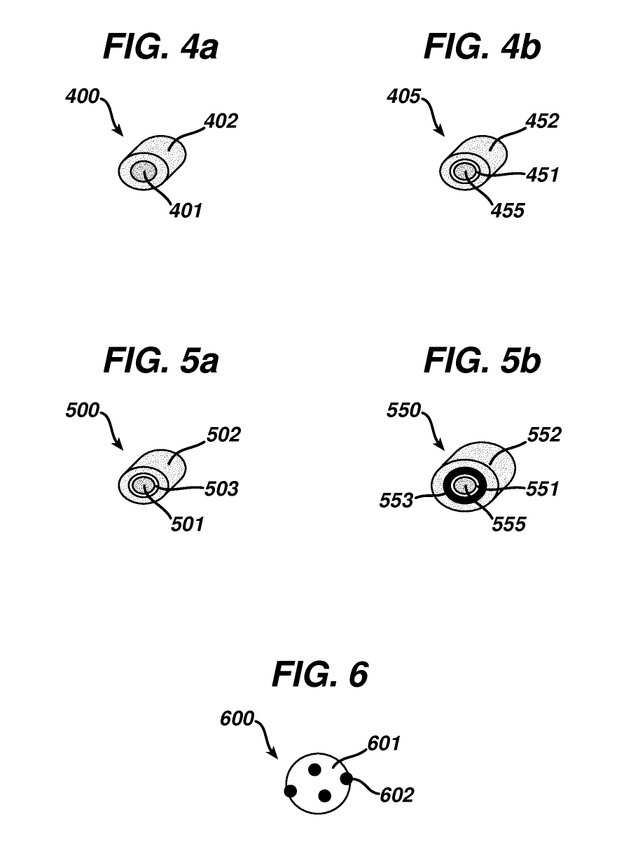

In one embodiment shown in FIG. 4a, to manufacture binary galvanic particulates, thin wires of a first conductive material 401 are coated with a second conductive material 402. The resulting coated wires are then shredded thus forming galvanic particulates 400.

In another embodiment shown in FIG. 4b, to manufacture binary galvanic particulates, a non-conductive substrate 455 which can be a polymer-based fiber or cellulose-based fiber or fiber made of starch or made of an edible composition such as sugar, starch, casein, gelatin, or a similar material, is first coated by the first conductive material 451 and then is over-coated by the second conductive material 452. The resulting coated fibers are then shredded thus forming galvanic particulates 405. This approach enables wide control of the quantities of the first and second conductive materials in each galvanic particulate.

### Tertiary Particulates

In one embodiment shown in FIG. 5a, a thin wire of a first conductive material 501 is first coated with conductive/resistive interlayer 503, which is then over coated with a second conductive material 502. The resulting coated wire is then shredded thus forming galvanic particulates 500.

In another embodiment, thin fibers or wires of a first conductive material are chemically treated to form a surface layer of modified first conductive material as a conductive/resistive interlayer, which is then over-coated by the second conductive material. Chemical treatments can include surface oxidation, conversion coating, blackening, etc.

In another embodiment shown in FIG. 5b, to manufacture tertiary galvanic particulates, a non-conductive substrate in the form of a fiber 555 is first coated by a first conductive material 551 and then coated by a conductive/resistive interlayer 553, which is then over-coated by a second conductive material 552. The resulting coated fiber is then shredded thus forming galvanic particulates 550.

### Multiphase Galvanic Particulates

In one embodiment, the invention provides a multiphase galvanic particulate comprising a dispersed phase comprising a second conductive material dispersed in a continuous phase comprising a first conductive material wherein both said first conductive material and said second conductive material are exposed on the surface of said particulate. The multiphase galvanic particulate comprises about 0.1 to about 99.9, preferably about 0.5 to about 60, more preferably about 0.5 to about 50, weight percent of said dispersed phase. The multiphase galvanic particulate is not a single-phase alloy, although it may contain one or more single-phase alloys either as dispersed phase(s) or the continuous phase. It comprises at least two heterogeneous phases.

The first conductive material serves as anode, and the second conductive material serves as cathode of the multiphase galvanic particulates. Both the anode and cathode are exposed on the surface of such galvanic particulates. The anode may be a single-phase or multi-phase alloy of the metals suitable as the first conductive material. The cathode may be a single-phase or multiphase alloy of the metals suitable as the second conductive material.

The size of the multiphase galvanic particulates is preferably larger than the size of the dispersed phase powder(s) to ensure that a majority of the multiphase galvanic particulates have at least one particle of the second conductive material. The size of the multiphase galvanic particles is generally in the range of about 0.1 to about 500 microns, for example less than about 200 microns or less than about 100 microns.

In one embodiment, the dispersed phase comprises copper metal and has a particle size of about 0.01-10 microns, and the continuous phase comprises zinc metal. The resulting material is processed to make multiphase galvanic particulates with a particle size of less than about 100 microns, the majority preferably having a particle size of about 1-50 microns.

In one embodiment, the dispersed phase has a melting point greater than about 950° C. In this embodiment, the second conductive material may, for example, be selected from the group consisting of copper, silver, gold, manganese, iron, and alloys thereof.

In another embodiment, the continuous phase has a melting point of less than about 750° C. In this embodiment, the first conductive material is selected from the group consisting of zinc, magnesium, aluminum, oxides thereof, halides thereof and alloys thereof.

The multiphase galvanic particulate may further comprise at least one additional dispersed phase comprising an additional conductive material, as defined above.

In another embodiment, the multiphase galvanic particulate comprises a conductive/resistive interlayer. For example, as described above, the conductive/resistive interlayer can be a modified form of the first or second conductive material, for example an oxide, halide or other salt, or another compound of the first or second conductive material. The conductive/resistive interlayer may also comprise a conversion coating on the interfacial surface of the first or second conductive material or other surface modification of the first or second conductive material.

The first conductive material serves as anode, and the second conductive material serves as cathode of the multiphase galvanic particulates. Both the anode and cathode are exposed on the surface of such galvanic particulates. The anode may be a single-phase or multi-phase alloy of the metals suitable as the first conductive material. The cathode may be a single-phase or multiphase alloy of the metals suitable as the second conductive material.

Multiphase galvanic particulates can be formed for example by the following process: (a) heat the first conductive material to a temperature above its melting point, so that it is either completely melted or partially melted for sintering or spray forming process, (b) disperse or mix particles, for example a fine powder, of the second conductive material into the molten first conductive material at a temperature above the melting point of the first conductive metal but below the melting point of the second conductive material, (c) micronize the resulting molten/solid mixture (e.g., by spray forming using a fine orifice with or without atomizer) to desirable small particle size and (d) cool the resulting particles to a lower or ambient temperature to form multiphase galvanic particulates.

Preferably, the aforementioned micronization or atomization processes are performed under a protective atmosphere (i.e., with inert gas such as argon, nitrogen, or carbon dioxide) or under a reducing atmosphere (e.g., hydrogen or its mixture with other inert gases).

Alternatively, step (c) may be carried out by: (i) cooling the molten/solid mixture to a lower or ambient temperature to form a solid composite, and (ii) mechanically micronizing (such as milling and/or shredding) the solid composite into multiphase galvanic particulates.

Such manufacturing methods, including the preferred spray forming process, are generally described Journal of Materials Processing Technology, Vol. 106, Issues 1-3, Pages 58-67 and “ASM Handbook Volume 7: Powder Metal Technologies and Applications” (by ASM International Handbook Committee, edited by Peter W. Lee, 1998), both incorporated herein by reference.

FIG. 6 depicts a multiphase galvanic particulate comprising a second conductive material (metal or alloy) 602 having a high melting point dispersed in first conductive material (metal or alloy) 601 having a low melting point. The second conductive material is also exposed on the surface of the first conductive material.

The aforementioned micronization (or atomization) methods for production of small particle size galvanic particulates are described in the ASM Handbook, infra, pages 31-109.

In another embodiment, multiphase galvanic particulates are manufactured by following steps: (a) heat a mixture of first, second, and optionally additional conductive materials at a desired weight or mole ratio to above all of their melting points to form a molten mixture, (b) micronize the molten mixture (e.g., by spray forming for example using a fine orifice with or without atomizer) to desired small particle size, and (c) cooling the micronized mixture droplets to a lower or ambient temperature, whereby phase separation occurs in the micronized mixture particles to form multiphase galvanic particulates comprising at least two alloy phases rich in different conductive materials. The phase rich in the first conductive material serves as an anode of the multiphase galvanic particulates, and the phase rich in the second conductive material serves as the cathode of the multiphase galvanic particulates.

Alternatively, step (b) may be carried out by: (i) cooling the molten mixture down to a lower or ambient temperature to form a solid composite, and (ii) mechanically micronizing (such as milling or shredding) the solid composite to yield multiphase galvanic particulates.

In another embodiment, the temperature during the melting process is carefully controlled and the process temperature is lowered gradually according to the metallurgy phase diagram for these conductive materials being used, so that the material with the higher melting point (usually the second conductive material) is solidified first as fine particles in the other (molten) material.

Alternatively, the molten mixture can be cast to form a solid composite with non-uniform domains of two conductive materials. The resulting composite is then processed by known techniques for particle size reduction such as milling, rolling, or shredding processes. The resulting powder is a multiphase galvanic particulate.

Three or more conductive materials can be processed by the above methods to manufacture multiphase galvanic particulates.

### Galvanic Particulates Formed by Multi-Layer Deposition on Substrates

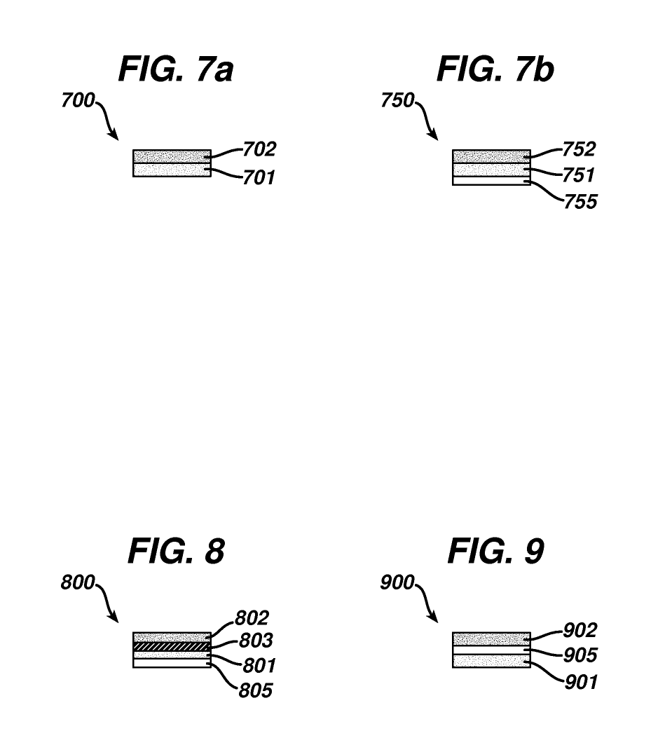

Referring now to FIG. 7a, in one embodiment galvanic particulate 700 comprises a layer of the first conductive material 701 deposited on a substrate (not shown), and a layer of the second conductive material 702 deposited on top of the first conductive material. Similarly, FIG. 7b depicts a galvanic particulate 750 comprising a layer of first conductive material 751 deposited on a substrate 755 and a layer of the second conductive material 752 deposited on top of the first conductive material.

The resulting two-layer material may have, for example a total thickness from about 1 micron to about 500 microns. The first and second conductive layers are lifted off of the substrate and broken down (e.g., shredded) into galvanic particulates of desirable size, for example into flakes ranging from about 5 microns to about 500 microns in maximum dimension. In one embodiment, both deposition steps are carried from a gas phase. In another embodiment, one deposition step is carried from a gas phase, and another deposition step is carried from a liquid phase. It should be noted that the sequence of deposition of the first conductive material and the second conductive material can be reversed.

In one embodiment, the substrate is a polymeric film. In another embodiment, the substrate is a soluble polymeric film, which is optionally removed by exposure to a suitable solvent, such as alcohol or water.

The substrate may have a conductivity, thickness, and porosity adapted for optimal discharge of galvanic particulates. In one embodiment the substrate contains pores, for example nano-pores or micro-pores. Such pores may be optionally filled by either first conductive material or second conductive material during deposition process, thus establishing electric connection between first conductive material and second conductive through pores.

Referring now to FIG. 8, in another embodiment, a layer of the first conductive material 801 is deposited on a substrate 805 and then a conductive/resistive interlayer 803 is formed on top of the first conductive material, for example by deposition or by chemical modification of a surface layer of the first conductive material, such as formatting its oxide as described above. Next, a second conductive material is deposited on top of the conductive/resistive interlayer. The resulting three-layer material, which may have a total thickness from about 1 micron to about 500 microns, is then shredded into particulate of desirable size, for example into rectangles with sizes ranging from about 5 microns to about 500 microns to form galvanic particulates.

Referring now to FIG. 9, in another embodiment, a layer of the first conductive material 901 is deposited on one side of a thin conducting, or non-conducting but porous polymeric substrate 905, and a layer of a second conductive material 902 is deposited on the other side of the same substrate.

The resulting three-layer material, which may have a total thickness from about 1 micron to about 500 microns, is then shredded into particulate of desirable size, for example into rectangles with sizes ranging from about 5 microns to about 500 microns to form galvanic particulates.

### Electroplating a Metal Foil

In this embodiment, a thin metal foil is coated or plated with one or more layers and then shredded or cut to form galvanic particulates, for example cut into squares having size of approximately 75×75 microns and thickness of approximately 25-50 microns. Binary galvanic particulates are formed from two-layer foils or three-layer foils containing first conductive material coated by second conductive material on both sides.

Tertiary galvanic particulates are manufactured by forming a layer of additional conductive material on at least one side of a foil consisting of first conductive material, and then coating the resulting material with the second conductive material.

Methods of foil coating are well known in the art, including continuous reel-to-reel foil electroplating, electroless plating, dip coating, and vacuum deposition coating.

### Electroplating of Particles Using Through-Mask Deposition

In one embodiment, galvanic particulates are formed on a reusable mandrel by through mask electro-deposition. A single use or reusable insulating polymeric sheet mask having multiple apertures is disposed on a conductive mandrel and the first and second conductive material are sequentially electrodeposited on the mandrel through mask apertures. The material of the mandrel is selected to have low adhesion to the electrodeposited first conductive material. The size of apertures can be from about 20 microns or less to about 500 microns or more, and the thickness of the mask can be from about 10 microns to about 500 microns. After the deposition is complete, the mask is lifted and the particles are washed off or scraped off of the mandrel and off the mask. Binary and tertiary galvanic particulates can be made by this process. Through mask electro-deposition processes are known in the art, and described, for example in U.S. Pat. Nos. 4,431,500; 4,921,583; 5,389,220; and 6,632,342.

### Clad Metal Foils

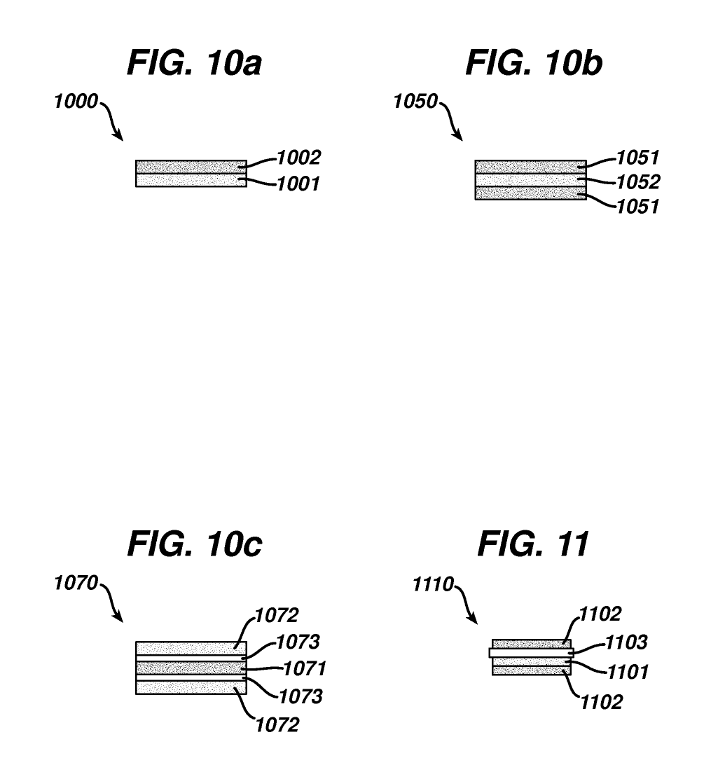

Referring now to FIG. 10a, in one embodiment, thin metal foils of first conductive material 1001 and the second conductive material 1002 are clad or bonded together, for example by cold rolling, optionally with a conductive/resistive interlayer. For example, the second conductive material 1052 may be located between two foils of the first conductive material 1051 as shown in FIG. 10b for galvanic particulate 1050. It should be noted the reverse order can also be prepared (i.e., with 1051 located between two layers of 1052). The resulting clad foils are then shredded into galvanic particulates, for example shredded into squares having size of approximately 75×75 microns and thickness of approximately 25-50 microns. Binary galvanic particulates are formed from two-layer foils or three-layer foils containing first conductive material clad with the second conductive material on both sides.

Tertiary galvanic particulates shown as 1070 in FIG. 10c are formed by cladding a first conductive material foil 1071 with a second conductive material foil 1072 while forming a conductive/resistive interlayer 1073 at the interface between the first and second conductive materials. Again, the location/placement for 1071 and 1072 can be reversed. The conductive/resistive interlayer may be formed by chemical modification of the first or second conductive materials, or both, for example by oxidative treatment, conversion coating, graphitization, plating, vapor deposition, or other means of surface modification as described infra. Methods of manufacturing of clad metallic foils, for example by cold rolling two foils together, are known in the art.

### Electroplating Particles to Manufacture Binary Galvanic Particulates

According to this method, particles comprising a first conductive material are immersed in an electro-plating bath having a cathode and an anode. The electrolyte contains an ionized form of a second conductive material, for example a soluble salt of a second conductive material. The electro-plating bath is continuously stirred, and particles in the bath form a suspension, with particles randomly approaching and contacting the cathode. Upon contact with the cathode particles are subjected to brief bursts of electro-deposition, which results in electro-deposition of the second conductive material. The deposition is non-uniform, leaving certain particles surface areas exposed. Coating of carbon particles or metal-based particles is possible, without regard to the activity of individual metals.

In another embodiment, slurry of particles consisting of the first conductive material is in contact with the cathode in the plating bath, with most active electro-deposition occurring in the top layer of the slurry. The resulting electro-deposition of the second conductive material from the electrolyte is non-uniform, leaving certain surface area of particles exposed.

### Porous Galvanic Particulates

In one embodiment, the galvanic particulate comprises a porous first conductive material, and a second conducive material impregnated in the pores of the porous first conductive material. The first conductive material may be a porous particle.

In one embodiment, a porous particle, for example a carbon microparticle, is impregnated with a solution of a salt of an active metal, for example a zinc salt. Optionally, an oxide of the active metal is formed, for example via reaction with a hydroxide solution, such as NaOH. After optional drying, the salt or oxide of the active metal is reduced by reaction with a reducing agent, such as carbon, which is present in the porous particle, hydrogen gas, CO, or other reducing agent. As a result, active metal deposits are formed in the pores and optionally on a part of the surface of the carbon particle.

In another embodiment, pores in a porous thin sheet or in a porous paper made of the first conductive material, such as carbon, are impregnated by an active metal or second conducive material as described above. The thin sheet is then shredded to form galvanic particulates of desired size.

In another embodiment, porous particles are further impregnated with an active therapeutic compound. Thus a combination treatment particle is formed combining galvanic properties and other therapeutic properties imparted by the active therapeutic compound.

### Other Substrates

As described above, the galvanic particulates may comprise substrates such as polymeric substrates. The substrates may also comprise such materials as waxes, polyesters, or similar materials, thin film substrates, fibers, soluble materials including soluble fibers, and edible materials including starch, casein, gelatin and similar materials. At least two conductive materials may be sequentially deposited on such materials, using deposition methods described herein. Such embodiments enable manufacturing of galvanic particulates with smaller loading of conductive materials and with conductive materials disposed as thin films.

### Application of Galvanic Particulates to Reusable Garments

In one embodiment, galvanic particulate are formed, as described above, on a polymeric core or on a polymeric substrate, the polymer being optionally water-soluble and having a low melting point. The galvanic particulates are then applied to a garment, for example as a brush-on, spray-on, or powder sprinkling. The galvanic particulates on the garment are then heated, for example by application of a hot iron, optionally hot iron with hot steam. The polymeric core or polymeric substrate exposed to elevated temperature melts and forms a bond with the fabric of the garment. The method enables the formation of an immobilized layer of galvanic particulates on the garment. The particles can be removed by washing or laundering the garment, and a fresh layer of the galvanic particulates can be applied.

In another embodiment, a thermally activated and optionally water-soluble adhesive powder is admixed to the galvanic particulates and the resulting mixture is applied to the garment through iron-on process.

In yet another embodiment, a suspension of galvanic particulates is formed in a suitable carrier, such as alcohol. The carrier also contains dissolved adhesive. The mixture is applied to the garment and the solvent is allowed to evaporate. Upon curing of the adhesive, which can be air-curable, light-curable, or drying-curable, a bond between galvanic particulates and the fabric of the garment is established.

### Combined Binary-Tertiary Galvanic Particulates

Referring now to FIG. 11, combination galvanic particulates comprising both slow-discharging tertiary galvanic features and fast-discharging binary galvanic features can be manufactured the methods described above. In one embodiment combination binary-tertiary galvanic particulates are formed by coating a conductive/resistive interlayer 1103 with a first conductive material 1101 on one side, and then over-coating the resulting material on both sides with a second conductive material 1102. Shredding the resulting film yields combination galvanic particulate. First conductive material and second conductive material are in direct contact on one side, forming a fast-discharging component, and first conductive material and second conductive material in indirect contact through the interlayer on the other side, forming the slow-discharging component.

In another embodiment, the galvanic particulates of the present invention may be coated with other materials to protect the galvanic materials from degradation during storage (e.g., oxidation degradation from oxygen and moisture), or to modulate the electrochemical reactions and to control the electric current generate when in use. The exemplary coating materials over the galvanic material(s) are inorganic or organic polymers, natural or synthetic polymers, biodegradable or bioabsorbable polymers, silica, glass, various metal oxides (e.g., oxide of zinc, aluminum, magnesium, or titanium) and other inorganic salts of low solubility (e.g., zinc phosphate). The coating methods are known in the art of metallic powder processing and metal pigment productions, such as those described by U.S. Pat. No. 5,964,936; U.S. Pat. No. 5,993,526; U.S. Pat. No. 7,172,812; US 20060042509A1 and US 20070172438.

In one embodiment, the galvanic particulates are stored in anhydrous forms, e.g., as a dry powder or immobilized in a fabric with binding agents, or as an essentially anhydrous non-conducting organic solvent composition (e.g., dissolved in polyethylene glycols, propylene glycol, glycerin, liquid silicone, and/or alcohol). In another embodiment, the galvanic particulates are embedded into the anhydrous carrier (e.g., inside a polymer) or coated onto a substrate (e.g., as a coating or in the coating layer of a healthcare product such as wound dressing or dental floss). In yet another embodiment, the galvanic particulates are encapsulated in compositions of microcapsules, liposomes, micelles, or embedded in the lipophilic phase of oil-in-water (O/W) or water-in-oil (W/O) types of emulsion systems (e.g., W/O lotion, W/O ointment, or O/W creams), as well as self-emulsifying compositions, in order to achieve self-life stability, retard the activation of the galvanic particulates, or prolong the action of galvanic particulates.

The galvanic particulates may also be compressed into tablets, incorporated into a polymer composition in a tablet coating film, incorporated into either hard or soft gelatin capsules, or incorporated waxy materials (e.g., as used in suppositories) or polymers (into bioabsorbable polymers as used in implant products or into biocompatible polymers as used in dental bracelets and toothbrushes). The coating (shell) materials used in the particulates may have an enteric property (e.g., being insoluble at acidic condition and only soluble when exposed to a medium with the pH value near or equal to neutral pH), or have a pH-sensitive permeability for the water and solute molecules and ions, or is biodegradable or bioabsorbable.

### Compositions and Products

The galvanic particulates have great versatility in applications, and can be used in many consumer and medical products for human and animal applications such as ingestible compositions (such as tablets and solutions), topical compositions (such as creams, lotions, gels, shampoos, cleansers, powders patches, bandages, and masks for application to the skin or mucosal membranes), garments (such as undergarments, underwear, bras, shirts, pants, pantyhose, socks, head caps, facial masks, gloves, and mittens), linens (such as towels, pillow covers or cases and bed sheets), and personal and medical products (such as sanitizing products for household and clinical settings, microcides for plants) and devices (such as toothbrushes, dental flosses, periodontal implants or inserts, orthodontic braces, joint wraps/supports, buccal patches, ocular inserts or implants such as contact lenses, nasal implants or inserts, and contact lens cleaning products, wound dressings, diapers, sanitary napkins, and wipes, tampons, rectal and vaginal suppositories, and galvanic particulate coatings or embedded in the surfaces of medical devices and other surfaces where the antimicrobial or other beneficial effects are desired). Many of such compositions and products are further discussed below.

In one embodiment, the galvanic particulates induce certain desirable biological responses that facilitate the treatment of the barrier membrane conditions (e.g., induced by the electric current passage through the skin, intestine, or mucosal membrane and/or enhancing the delivery of an active agent). In one embodiment, the galvanic particulates provide multiple mechanism of actions to treat conditions, such as to enhance delivery of an active agents by iontophoresis and/or electro-osmosis as well as provide electric stimulation to treat the contacted tissue (e.g., to increase blood circulation or other benefits).

What is meant by an “active agent” is a compound (e.g., a synthetic compound or a compound isolated from a natural source) that has a cosmetic or therapeutic effect on the skin or other barrier membrane and the surrounding tissues (e.g., a material capable of exerting a biological effect on a human body) such as therapeutic drugs or cosmetic agents. Examples of such therapeutic drugs include small molecules, peptides, proteins, nucleic acid materials, and nutrients such as minerals and extracts. The amount of the active agent in the carrier will depend on the active agent and/or the intended use of the composition or product. In one embodiment, the composition containing the galvanic particulates further contains a safe and effective amount of an active agent, for example, from about 0.001 percent to about 20 percent, by weight, such as from about 0.01 percent to about 10 percent, by weight, of the composition.

The galvanic particulates can be combined with an active agent (such as antimicrobial agents, anti-inflammatory agents, and analgesic agents) to enhance or potentiate the biological or therapeutic effects of that active agent. In another embodiment, the galvanic particulates can also be combined with other substances to enhance or potentiate the activity of the galvanic particulates. Substances that can enhance or potentiate the activity of the galvanic particulates include, but are not limited to, organic solvents (such as alcohols, glycols, glycerin, polyethylene glycols and polypropylene glycol), surface active agents (such as nonionic surfactants, zwitterionic surfactants, anionic surfactants, cationic surfactants and polymeric surfactants), and water-soluble polymers. For example, the galvanic particulates of the present invention can form conjugates or composites with synthetic or natural polymers including by not limited to proteins, polysaccharides, hyaluronic acid of various molecular weight, hyaluronic acid analogs, polypeptides, and polyethylene glycols.

In one embodiment, the composition contains a chelator or chelating agent. Examples of chelators include, but are not limited to, amino acids such as glycine, lactoferrin, edetate, citrate, pentetate, tromethamine, sorbate, ascorbate, deferoxamine, derivatives thereof, and mixtures thereof. Other examples of chelators useful are disclosed in U.S. Pat. No. 5,487,884 and PCT Publication Nos. 91/16035 and 91/16034.

### Methods of Using Galvanic Particulates

In one embodiment, the galvanic particulates are used to provide the therapeutic electric stimulation effects by applying the galvanic particulates directly to a target human tissue in need such a therapeutic treatment (e.g., either topically or inside the body), including soft tissues (e.g., the skin, mucosa, epithelium, wound, eye and its surrounding tissues, cartilage and other soft musculoskeletal tissues such as ligaments, tendons, or meniscus), hard tissues (e.g., bone, teeth, nail matrix, or hair follicle), and soft tissue-hard tissue conjunctions (e.g., conductive tissues around periodontal area involved teeth, bones or soft tissue of the joint).

Application of galvanic particulates can be for the purpose of treating tissue for therapeutic effects including, but not limited to: antimicrobial effects (e.g., antibacterial, antifungal, antiviral, and anti-parasitic effects); anti-inflammation effects including effects in the superficial or deep tissues (e.g., reduce or elimination of soft tissue edema or redness); elimination or reduction of pain, itch or other sensory discomfort (e.g., headache, sting or tingling numbness); regeneration (i.e., replacement or regrowth of lost or damaged tissue or tissue components to restore original function and appearance thereof), rejuvenation or healing enhancement of both soft and hard tissues; modulation of stem cell differentiation and tissue development such as modulation of tissue growth (e.g., enhancing growth rate of the nail or regrowth of hair loss due to alopecia) or increase soft tissue volume (e.g., increasing collagen or elastin in soft tissues such as the skin or lips); increasing adepocyte metabolism or improving body appearance (e.g., effects on body contour or shape); and increasing circulation of blood or lymphocytes.

One skilled in the art will recognize that, both in vivo and in vitro trials using suitable, known and generally accepted cell and/or animal models are predictive of the ability of an ingredient, composition, or product to treat or prevent a given condition. One skilled in the art will further recognize that human clinical trials including first-in-human, dose ranging and efficacy trials, in healthy patients and/or those suffering from a given condition or disorder, may be completed according to methods well known in the clinical and medical arts.

### Ingestible Compositions

The ingestible compositions according to the invention are suitable for ingesting by a mammal, such as a human, in need of treatment. In one embodiment, the compositions contain a safe and effective amount of (i) the galvanic particulates and (ii) a pharmaceutically-acceptable carrier.

In one embodiment, the ingestible compositions herein contain, per dosage unit (e.g., tablet, capsule, powder, injection, teaspoonful and the like) an amount of the galvanic particulates and/or active agent necessary to deliver an effective dose as described above. In one embodiment, the ingestible compositions herein contains, per unit dosage unit of from about 1 mg to about 5 g of the galvanic particulates and/or active agent, such as from about 50 mg to about 500 mg, and may be given at a dosage of from about 1 mg/kg/day to about 1 g/kg/day, such as from about 50 to about 500 mg/kg/day. The dosages, however, may be varied depending upon the requirement of the patients, the severity of the condition being treated, and the galvanic particulates being employed. The use of either daily administration or post-periodic dosing may be employed. In one embodiment, these compositions are in unit dosage forms from such as tablets, pills, capsules, powders, granules, solutions or suspensions, and drops.

In one embodiment, the compositions are provided in the form of tablets, such as those containing 1, 5, 10, 25, 50, 100, 150, 200, 250, 500, and/or 1000 milligrams of the galvanic particulates and/or active agent for the symptomatic adjustment of the dosage to the patient to be treated. The composition may be administered on a regimen of 1 to 4 times per day. Advantageously, the compositions may be administered in a single daily dose, or the total daily dosage may be administered in divided doses of two, three or four times daily.

Optimal dosages to be administered may be readily determined by those skilled in the art, and will vary with the particular galvanic particulates and/or active agent used, the mode of administration, the strength of the preparation, and the advancement of the disease/condition being treated. In addition, factors associated with the particular patient being treated, including patient age, weight, diet and time of administration, will result in the need to adjust dosages.

Ingestible compositions containing one or more types of the galvanic particulates of the invention described herein can be prepared by intimately mixing the galvanic particulates with a pharmaceutically-acceptable carrier according to conventional pharmaceutical compounding techniques. The carrier may take a wide variety of forms depending upon the type of formulation. Thus for liquid preparations such as suspensions, elixirs and solutions, suitable carriers and additives include but not limited to water, glycols, alcohols, silicones, waxes, flavoring agents, buffers (such as citrate buffer, phosphate buffer, lactate buffer, gluconate buffer), preservatives, stabilizers, coloring agents and the like; and for solid preparations, such as powders, capsules and tablets, suitable carriers and additives include starches, sugars, diluents, granulating agents, lubricants, binders, disintegrating agents and the like. Solid oral preparations may also be coated with substances such as sugars, soluble polymer film, and insoluble-but-solute permeable polymer film. Oral preparation may also be coated with enteric coating, which is not soluble in the acidic stomach environment but will dissolve in the intestine as the pH becomes neutral so as to modulate major site of galvanic particulate action. For product storage and stability, the galvanic particulates should preferably be kept in an anhydrous or relatively non-conductive phase or compartment.

For preparing solid compositions such as tablets, the galvanic particulates are mixed with a pharmaceutically-acceptable carrier, e.g. conventional tableting ingredients such as corn starch, lactose, sucrose, sorbitol, talc, stearic acid, magnesium stearate, dicalcium phosphate or gums, and other pharmaceutically-acceptable diluents, to form a solid preformulation composition containing a homogeneous mixture of the galvanic particulates. When referring to these preformulation compositions as homogeneous, it is meant that the galvanic particulates is dispersed evenly throughout the composition so that the composition may be readily subdivided into equally effective dosage forms such as tablets, pills and capsules. This solid preformulation composition may then be subdivided into unit dosage forms of the type described above. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

### (a) Gastro-Intestinal Disorder Treatment Ingestible Compositions

In one embodiment, ingestible compositions containing the galvanic particulates are used for the treatment of gastrointestinal disorders, such as ulcers, diarrhea, and gastrointestinal pain.

In one embodiment, the galvanic particulates can be combined with active agents known to treat diarrhea which include, but are not limited to: bismuths (such as Bismuth Subsalicylate), Loperamide, Simethicone, Nitazoxanide, Ciprofloxacin, and Rifaximin, salts and prodrugs (such as esters) thereof.

In one embodiment, the galvanic particulates can be combined with active agents known to treat gastric ulcers which include, but are not limited to: Lansoprazole, Naproxen, Esomeprazole, Famotidine, Nizatidine, Ranitidine, and Omeprazole, and salts and prodrugs thereof.

In one embodiment, the galvanic particulates can be combined with active agents known to treat intra-abdominal infections which include, but are not limited to: Moxifloxacin, Ciprofloxacin, Ceftazidime, Gentamicin, Ertapenem; Cefepime, Cefoxitin, Cilastatin, Imipenem; Ceftriaxone, Clavulanate, and Ticarcillin, and salts and prodrugs thereof

### (b) Pain Treating Ingestible Compositions

In one embodiment, ingestible compositions containing the galvanic particulates are used for treatment of pain (such as throat pain). Oral dosage forms can be in the forms of, but not limited to, lozenges or liquids. Galvanic particulates can be combined with active agents known to treat sore throat, which include, but are not limited to: Acetaminophen, Dextromethorphan, Pseudoephedrine, Chlorpheniramine, Pseudoephedrine, Guaifenesin, Doxylamine, Zinc, and Ibuprofen, and salts and prodrugs thereof

### (c) Oral Supplement Ingestible Compositions

In one embodiment, ingestible compositions containing the galvanic particulates are used as oral supplements or complements to oral supplements. Oral dosage forms can be in the forms of, but not limited to, lozenges, tablets, caplets, powders, or liquids. Galvanic particulates can be combined with oral supplements of vitamins and minerals, which include, but are not limited to: Dibasic Calcium Phosphate, Magnesium Oxide, Potassium Chloride, Microcrystalline Cellulose, Ascorbic Acid (Vit. C), Ferrous Fumarate, Calcium Carbonate, dl-Alpha Tocopheryl Acetate (Vit. E), Acacia, Ascorbyl Palmitate, Beta Carotene, Biotin, BHT, Calcium Pantothenate, Calcium Stearate, Chromic Chloride, Citric Acid, Crospovidone, Cupric Oxide, Cyanocobalamin (Vit. B₁₂), Ergocalciferol (Vit. D), Folic Acid, Gelatin, Hypromellose, Lutein, Lycopene, Magnesium Borate, Magnesium Stearate, Manganese Sulfate, Niacinamide, Nickelous Sulfate, Phytonadione (Vit. K), Potassium Iodide, Pyridoxine Hydrochloride (Vit. B₆), Riboflavin (Vit. B₂), Silicon Dioxide, Sodium Aluminum Silicate, Sodium Ascorbate, Sodium Benzoate, Sodium Borate, Sodium Citrate, Sodium Metavanadate, Sodium Molybdate, Sodium Selenate, Sorbic Acid, Stannous Chloride, Sucrose, Thiamine Mononitrate (Vit. B₁), Titanium Dioxide, Tribasic Calcium Phosphate, Vitamin A Acetate (Vit. A), and Zinc Oxide., and salts and prodrugs thereof.

In addition, in one embodiment, the metal components of the galvanic particulates can serve as mineral supplements generated in situ, e.g. zinc metal converted to zinc ion in situ.

### Topical Skin Compositions

In one embodiment, topical compositions useful in the present invention involve compositions containing the galvanic particulates that are suitable for administering to mammalian skin, such as human skin. In one embodiment, the compositions contain a safe and effective amount of (i) the galvanic particulates and (ii) a pharmaceutically-acceptable carrier.

The compositions may be made into a wide variety of products that include but are not limited to leave-on products (such as lotions, creams, gels, sticks, sprays, and ointments), skin cleansing products (such as liquid washes, solid bars, and wipes), hair products (such as shampoos, conditioners, sprays, and mousses), shaving creams, film-forming products (such as masks), make-up (such as foundations, eye liners, and eye shadows), deodorant and anti-perspirant compositions, and the like. These product types may contain several types of pharmaceutically-acceptable carrier forms including, but not limited to solutions, suspensions, emulsions such as microemulsions and nanoemulsions, gels, and solids carrier forms. Other product forms can be formulated by those of ordinary skill in the art.

In one embodiment, the composition or product is used for the treatment of skin diseases and conditions. Examples of such treatments include, but are not limited to: the treatment of acne (e.g., blackheads and whiteheads), rosacea, nodule-cystic, and other microbial infections of the skin; reduction the visible signs of skin aging (e.g., wrinkles, sagging, sallowness, and age-spots); firming the skin; enhancing the elasticity of the skin; folliculitis and pseudo-folliculitis barbae; sebum regulation (e.g., sebum reduction or oily/shining skin appearance inhibition or control); pigmentation regulation (e.g., reduction of hyperpigmentation such as freckles, melasma, actinic and senile lentigines, age-spots, post-inflammatory hypermelanosis, Becker's naevus, and facial melanosis or enhancing the pigmentation of light skin); hair growth retardation (e.g., skin on the leg) or hair stimulation (e.g., to the scalp); and the treatment of dermatitis (e.g., atopic, contact, or seborrheic dermatitis), dark circles under the eye, stretch marks, cellulite, excessive sweating (e.g., hyperhidrosis), and/or psoriasis.

### (a) Topical Anti-Acne/Anti-Rosacea Compositions

In one embodiment, the composition or product contains an anti-acne and/or anti-rosacea active agent. Examples of anti-acne and anti-rosacea agents include, but are not limited to: retinoids such as tretinoin, isotretinoin, motretinide, adapalene, tazarotene, azelaic acid, and retinol; salicylic acid; benzoyl peroxide; resorcinol; sulfur; sulfacetamide; urea; antibiotics such as tetracycline, clindamycin, metronidazole, and erythromycin; anti-inflammatory agents such as corticosteroids (e.g., hydrocortisone), ibuprofen, naproxen, and hetprofen; and imidazoles such as ketoconazole and elubiol; and salts and prodrugs thereof. Other examples of anti-acne active agents include essential oils, alpha-bisabolol, dipotassium glycyrrhizinate, camphor, β-glucan, allantoin, feverfew, flavonoids such as soy isoflavones, saw palmetto, chelating agents such as EDTA, lipase inhibitors such as silver and copper ions, hydrolyzed vegetable proteins, inorganic ions of chloride, iodide, fluoride, and their nonionic derivatives chlorine, iodine, fluorine, and synthetic phospholipids and natural phospholipids such as Arlasilk™ phospholipids CDM, SV, EFA, PLN, and GLA (Uniqema, ICI Group of Companies, Wilton, UK).

### (b) Topical Anti-Aging Compositions

In one embodiment, the composition or product contains an anti-aging agent.

Examples of suitable anti-aging agents include, but are not limited to: inorganic sunscreens such as titanium dioxide and zinc oxide; organic sunscreens such as octyl-methoxy cinnamates; retinoids; dimethylaminoathanol (DMAE), copper containing peptides, vitamins such as vitamin E, vitamin A, vitamin C, and vitamin B and vitamin salts or derivatives such as ascorbic acid di-glucoside and vitamin E acetate or palmitate; alpha hydroxy acids and their precursors such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucoheptonic acid, glucoheptono 1,4-lactone, gluconic acid, gluconolactone, glucuronic acid, glucuronolactone, isopropyl pyruvate, methyl pyruvate, mucic acid, pyruvic acid, saccharic acid, saccaric acid 1,4-lactone, tartaric acid, and tartronic acid; beta hydroxy acids such as beta-hydroxybutyric acid, beta-phenyl-lactic acid, and beta-phenylpyruvic acid; tetrahydroxypropyl ethylene-diamine, N,N,N′,N′-Tetrakis(2-hydroxypropyl)ethylenediamine (THPED); and botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, nice, and safflower; and salts and prodrugs thereof

### (c) Topical Depigmentation Compositions

In one embodiment, the composition or product contains a depigmentation agent. Examples of suitable depigmentation agents include, but are not limited to: soy extract; soy isoflavones; retinoids such as retinol; kojic acid; kojic dipalmitate; hydroquinone; arbutin; transexamic acid; vitamins such as niacin and vitamin C; azelaic acid; linolenic acid and linoleic acid; placertia; licorice; and extracts such as chamomile and green tea; and salts and prodrugs thereof

### (d) Topical Antipsoriatic Compositions

In one embodiment, the composition or product contains an antipsoriatic active agent. Examples of antipsoriatic active agents (e.g., for seborrheic dermatitis treatment) include, but are not limited to, corticosteroids (e.g., betamethasone dipropionate, betamethasone valerate, clobetasol propionate, diflorasone diacetate, halobetasol propionate, triamcinonide, dexamethasone, fluocinonide, fluocinolone acetonide, halcinonide, triamcinolone acetate, hydrocortisone, hydrocortisone verlerate, hydrocortisone butyrate, aclometasone dipropionte, flurandrenolide, mometasone furoate, methylprednisolone acetate), methotrexate, cyclosporine, calcipotriene, anthraline, shale oil and derivatives thereof, elubiol, ketoconazole, coal tar, salicylic acid, zinc pyrithione, selenium sulfide, hydrocortisone, sulfur, menthol, and pramoxine hydrochloride, and salts and prodrugs thereof

### (e) Other Ingredients

In one embodiment, the composition or product contains a plant extract as an active agent. Examples of plant extracts include, but are not limited to, feverfew, soy, glycine soja, oatmeal, wheat, aloe vera, cranberry, witch-hazel, alnus, arnica, artemisia capillaris, asiasarum root, birch, calendula, chamomile, cnidium, comfrey, fennel, galla rhois, hawthorn, houttuynia, hypericum, jujube, kiwi, licorice, magnolia, olive, peppermint, philodendron, salvia, sasa albo-marginata, natural isoflavonoids, soy isoflavones, and natural essential oils.

In one embodiment, the composition or product contains a buffering agent such as citrate buffer, phosphate buffer, lactate buffer, gluconate buffer, or gelling agents, thickeners, or polymers.

In one embodiment, the composition or product contains a fragrance effective for reducing stress, calming, and/or affecting sleep such as lavender and chamomile.

### Topical Mucosal Compositions

In one embodiment, topical compositions useful in the present invention involve compositions containing the galvanic particulates that are suitable for administering to the mucosal membrane, such as human oral, rectal, and vaginal mucosal membranes. In one embodiment, the compositions contain a safe and effective amount of (i) the galvanic particulates and (ii) a pharmaceutically-acceptable carrier.

The compositions may be made into a wide variety of products for application on mucosa, including but not limited to vaginal creams, tampons, suppositories, floss, mouthwash, toothpaste. Other product forms can be formulated by those of ordinary skill in the art.

In one embodiment, the composition or product is used for the treatment of a mucosal membrane conditions. Examples of such treatments include, but are not limited to, treatment of vaginal candidiasis and vaginosis, genital and oral herpes, cold sore, canker sore, oral hygiene, periodontal disease, teeth whitening, halitosis, prevention of biofilm attachment, and other microbial infections of the mucosa.

The galvanic particulates can be incorporated into compositions for the treatment of candidiasis with actives such as, but not limited to: Tioconazole; Clotrimazole; and Nystatin.

The galvanic particulates can be incorporated into compositions for the treatment of bacterial vaginosis with actives such as, but not limited to, Clindamycin Hydrochloride and Metronidazole.

The galvanic particulates can be incorporated into compositions for the treatment of periodontal disease with actives such as, but not limited to minocycline.

### Compositions for Treatment of Wounds and Scars

In one embodiment, the galvanic particulates are incorporated into wound dressings and bandages to provide electric therapy for healing enhancement and scar prevention. In one embodiment, the wound exudation fluid and/or wound cleansing solution serves to activate a galvanic particulate containing wound dressing/bandage to (i) deliver active agents pre-incorporated in the wound dressing/bandage and/or (ii) to generate electrochemically beneficial metal ions followed with delivery of the beneficial metal ions into the wound and/or (iii) treat the wound with therapeutic electric current which may increase blood circulation, stimulate tissue immune response, and/or suppress tissue inflammation, which may lead to accelerated healing and reduced scarring.

In one embodiment, the composition or product contains an active agent commonly used as for topical wound and scar treatment, such as topical antibiotics, anti-microbials, wound healing enhancing agents, topical antifungal drugs, anti-psoriatic drugs, and anti-inflammatory agents.

Examples of antifungal drugs include but are not limited to miconazole, econazole, ketoconazole, sertaconazole, itraconazole, fluconazole, voriconazole, clioquinol, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, and their pharmaceutically acceptable salts and prodrugs. In one embodiment, the antifungal drug is an azole, an allylamine, or a mixture thereof.

Examples of antibiotics (or antiseptics) include but are not limited to mupirocin, neomycin sulfate bacitracin, polymyxin B, l-ofloxacin, tetracyclines (chlortetracycline hydrochloride, oxytetracycline—10 hydrochloride and tetrachcycline hydrochloride), clindamycin phsphate, gentamicin sulfate, metronidazole, hexylresorcinol, methylbenzethonium chloride, phenol, quaternary ammonium compounds, tea tree oil, and their pharmaceutically acceptable salts and prodrugs.

Examples of antimicrobials include but are not limited to salts of chlorhexidine, such as Iodopropynyl butylcarbamate, diazolidinyl urea, chlorhexidene digluconate, chlorhexidene acetate, chlorhexidene isethionate, and chlorhexidene hydrochloride. Other cationic antimicrobials may also be used, such as benzalkonium chloride, benzethonium chloride, triclocarbon, polyhexamethylene biguanide, cetylpyridium chloride, methyl and benzothonium chloride. Other antimicrobials include, but are not limited to: halogenated phenolic compounds, such as 2,4,4′,-trichloro-2-hydroxy diphenyl ether (Triclosan); parachlorometa xylenol (PCMX); and short chain alcohols, such as ethanol, propanol, and the like. In one embodiment, the alcohol is at a low concentration (e.g., less than about 10% by weight of the carrier, such as less than 5% by weight of the carrier) so that it does not cause undue drying of the barrier membrane.

Examples of anti-viral agents for viral infections such as herpes and hepatitis, include, but are not limited to, imiquimod and its derivatives, podofilox, podophyllin, interferon alpha, acyclovir, famcyclovir, valcyclovir, reticulos and cidofovir, and salts and prodrugs thereof.

Examples of anti-inflammatory agent, include, but are not limited to, suitable steroidal anti-inflammatory agents such as corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and salts are prodrugs thereof. In one embodiment, the steroidal anti-inflammatory for use in the present invention is hydrocortisone. A second class of anti-inflammatory agents which is useful in the compositions of the present invention includes the nonsteroidal anti-inflammatory agents.

Examples of wound healing enhancing agent include recombinant human platelet-derived growth factor (PDGF) and other growth factors, ketanserin, iloprost, prostaglandin E₁ and hyaluronic acid, scar reducing agents such as mannose-6-phosphate, analgesic agents, anesthetics, hair growth enhancing agents such as minoxadil, hair growth retarding agents such as eflornithine hydrochloride, antihypertensives, drugs to treat coronary artery diseases, anticancer agents, endocrine and metabolic medication, neurologic medications, medication for cessation of chemical additions, motion sickness, protein and peptide drugs.

### Treatment of Microbial Infections of the Body

In one embodiment, the galvanic particulates are used, with or without other antifungal active agents, to treat and prevent fungal infections (e.g., dermatophytes such as trichophyton mentagrophytes), including, but not limited to, onychomycosis, sporotrichosis, tinea unguium, tinea pedis (athlete's foot), Tinea cruris (jock itch), tinea corporis (ringworm), tinea capitis, tinea versicolor, and candida yeast infection-related diseases (e.g., candida albicans) such as diaper rash, oral thrushm, cutaneous and vaginal candidiasis, genital rashes, Malassezia furfur infection-related diseases such as Pityriasis versicolor, Pityriasis folliculitis, seborrhoeic dermatitis, and dandruff.

In another embodiment, the galvanic particulates are used, with or without other antibacterial active agents, to treat and prevent bacterial infections, including, but not limited to, acne, cellulitis, erysipelas, impetigo, folliculitis, and furuncles and carbuncles, as well as acute wounds and chronic wounds (venous ulcers, diabetic ulcers and pressure ulcers).

In another embodiment, the galvanic particulates are used, with or without other antiviral active agents, to treat and prevent viral infections of the skin and mucosa, including, but not limited to, molluscum contagiosum, warts, herpes simplex virus infections such as cold sores, kanker sores and genital herpes.

In another embodiment, the galvanic particulates are used, with or without other antiparasitic active agents, to treat and prevent parasitic infections, including, but not limited to, hookworm infection, lice, scabies, sea bathers' eruption and swimmer's itch.

In one embodiment, the particulates are administered to help treat ear infections (such as those caused by streptococcus oneumoniae), rhinitis and/or sinusitis (such as caused by Haemophilus influenzae, Moraxella catarrhalis, Staphylococcus aureus and Streptococcus pneumoniae), and strep throat (such as caused by Streptococcus pyogenes).

In one embodiment, the particulates are ingested by an animal (e.g., as animal feed) or a human (e.g., as a dietary supplement) to help prevent outbreaks of food borne illnesses (e.g., stemming from food borne pathogens such as Campylobacter jejuni, Listeria monocytogenes, and Salmonella enterica).

### Drug Resistant Microorganisms

In one embodiment, the invention features a method of killing pathogens drug resistant microorganisms by contacting the microorganism with a composition containing a galvanic particulate including a first conductive material and a second conductive material, wherein both the first conductive material and the second conductive material are exposed on the surface of the particulate, and wherein the difference of the standard potentials of the first conductive material and the second conductive material is at least about 0.2 V. In one embodiment, the particle size of said particulate is from about 10 nanometers to about 1000 micrometers, such as from about 1 micrometer to about 100 micrometers. In one embodiment, the second conductive material is from about 0.01 percent to about 10 percent, by weight, of the total weight of the particulate. In one embodiment, the drug resistant microoriganism is a bacteria, such as MRSA and VRE. In one embodiment, the particulates are administered via a nasal spray, rinse solution, or ointment.

### Nail Treatment Composition

The galvanic particulates can also be used to stimulate nail growth, enhance nail strength, and reduce nail infection or discoloration. The galvanic particulates can be incorporated into compositions for the treatment of onychomychosis with actives such as, but not limited to: miconazole, econazole, ketoconazole, sertaconazole, itraconazole, fluconazole, voricoriazole, clioquinol, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, and their pharmaceutically acceptable salts and prodrugs. Galvanic particulates can be incorporated into compositions for improving the look and feel of nails with ingredients such as, but not limited to: biotin, calcium panthotenate, tocopheryl acetate, panthenol, phytantriol, cholecalciferol, calcium chloride, Aloe Barbadensis (Leaf Juice), silk Protein, soy protein, hydrogen peroxide, carbamide peroxide, green tea extract, acetylcysteine and cysteine.

### Tissue-Augmentation and Tissue Engineering Applications

In one embodiment, the galvanic particulates can be used to reduce the visibility of skin facial wrinkles, reduce atrophy, or increase facial dermal and subdermal volumes and lip volume. The galvanic particulates may be used either alone or in conjunction with other components well known in the art, such as subcutaneous fillers, implants, periodontal implants, intramuscular injections, and subcutaneous injections, such as bio-absorbable polymers. For example, the galvanic particulates may be used in conjunction with collagen, hyaluronic acid, poly-L-Lactic acid, and/or Calcium hydroxyl apatite injections.

In another embodiment, the galvanic particulates can be incorporated into biodegradable scaffolds for tissue engineering and organ printing with techniques known in the art. It is known that electric stimulation can stimulate and promote differentiation, proliferation, and migration of biological cells (e.g. stem cells) to grow, repair and renew tissue. A recent publication describes the use of electricity in tissue engineering (Part et al., “Electrical stimulation and extra-cellular matrix remodeling of C2C12 cells cultured on collagen scaffolds”, J. Tissue Engineering and Regenerative Medicine 2(5) 2008, pages 279-287), and U.S. Patent Application 2005/0112759 A1 discloses the application of electrical stimulation for functional tissue engineering in vitro and in vivo. These references are incorporated by reference in their entirety. The galvanic particulates of the present invention can be used to provide electric stimulation in engineering for tissue regeneration in vitro, and more importantly ex vivo and in vivo because the galvanic particulates can readily be incorporated or manufactured into the tissue scaffold to be implanted into patients.

### Transdermal Drug Delivery Patches

In one embodiment, the galvanic particulates are incorporated into transdermal drug delivery patches to enhance active agent penetration into the skin by iontophoresis and to reduce skin irritation by electric stimulation and electrically generated beneficial ions, such as zinc ions.

Examples of such active agents include peptides, polypeptides, proteins, and nucleic acid materials comprising DNA; and nutrients. Examples of polypeptide and protein active agents include thyrotropin-releasing hormone (TRH), vasopressin, gonadotropin-releasing hormone (GnRH or LHRH), melanotropin-stimulating hormone (MSH), calcitonin, growth hormone releasing factor (GRF), insulin, erythropoietin (EPO), interferon alpha, interferon beta, oxytocin, captopril, bradykinin, atriopeptin, cholecystokinin, endorphins, nerve growth factor, melanocyte inhibitor-I, gastrin antagonist, somatotatin, encephalins, melatonin, vaccines, botox (Botulinum neurotoxins), cyclosporin and its derivatives (e.g., biologically active fragments or analogs). Other active agents include anesthetics; analgesics (e.g., fentanyl and salts thereof such fentanyl citrate); drugs for treating psychiatric disorders, epilepsies, and migraine; drugs for stopping drug additions and abuses; anti-inflammatory agents; drugs to treat hypertension, cardiovascular diseases, gastric acidity and ulcers; drugs for hormone replacement therapies and contraceptives such as estrogens and androgens; antibiotics, antifungals, antiviral and other antimicrobial agents; antineoplastic agents, immunosuppressive agents and immunostimulants; and drugs acting on blood and the blood forming argans including hematopoietic agents and anticoagulants, thrombolytics, and antiplatelet drugs. Other active agents that can be delivered into the body using such patches include vaccines for various diseases, such as those for influenza, AIDS, hepatitis, measles, mumps, rubella, rabies, rubella, avercella, tetanus, hypogammaglobulinemia, Rh disease, diphtheria, botulism, snakebite, back widow bite and other insect bite/sting, idiopathic thrombocytopenic purpura (ITP), chronic lymphocytic leukemia, cytomegalovirus (CMV) infection, acute renal rejection, oral polio, tuberculosis, pertussis, Haemophilus b, Pneumococcus, and Staphylococcus aureus.

Incorporation onto Substrates

The galvanic particulates can be incorporated onto fibers, nonwovens, hydrocolloids, adhesives, films, polymers, and other substrates. Products include but are not limited to dental floss, toothbrushes, sanitary napkins, tampons, bandages, wound dressings, casts, hairbrushes, and clothing. In one embodiment, the galvanic particulates are in contact with the tissue interface. Methods of applying the galvanic particulates on the substrates include electrostatic spray coating, mechanical sieving, co-extrusion, adhesive spraying,

The galvanic particulates may also be coated onto medical implants or surgical tools (e.g., to help prevent infections).

Sulfhydryl Compound Coating on a Galvanic Particulate for Improved Performance

One embodiment of the present invention provides a galvanic particulate comprising a coating comprising a compound having at least one sulfhydryl (SH) functional group. The sulfhydryl group links the compound to the surface of a galvanic particulate, which provides various beneficial effects. Advantages include, but are not limited to, (a) regulating generation of galvanic electricity; (b) enhancing tissue biocompatibility of the galvanic particulates, (c) improving the stability of the galvanic particulates; (d) enabling attachment of other chemical and biochemical moieties to the coating on the galvanic particulate surface by chemical bonds such as covalent bonds with the sulfhydryl compound for intended biological effects.

The term “sulfhydryl compound” includes: (a) thio-compounds with one or more sulfhydryl functional groups capable of reacting with the metallic surface of galvanic particulates of the present invention, and (b) thio-containing amino acids and their derivatives.

Thio-compounds with one or more sulfhydryl functional groups capable of reacting with the surface of galvanic partiulates include but are not limited to thioglycolic acid and its salts, such as glycolates of calcium, sodium, strontium, potassium, ammonium, lithium, magnesium, and other metal salts; thioethylene glycol, thioglycerol, thioethanol, as well as thioactic acid, thiosalicylic acid and their salts.

Thio-containing amino acids and their derivatives may be selected from the group consisting of L-cysteine, D-cysteine, DL-cysteine, N-acetyl-L-cysteine, DL-homocysteine, L-cysteine methyl ester, L-cysteine ethyl ester, N-carbamoyl cysteine, glutathion, and cysteamine

The preferred thio-compounds with one or more sulfhydryl functional groups capable of reacting with galvanic particulates are the calcium and sodium salts of thioglycolic acid.

The preferred thio-containing amino acids and their derivatives are L-cysteine and N-acetyl-L-cysteine.

In general, any compounds containing sulfhydryl functional group(s) capable of reacting with the metallic surface of the galvanic particulates can be used to produce the sulfhydryl coating on the galvanic particulates of the present invention.

One embodiment of the invention employs a sulfhydryl containing amino acid or a derivative thereof, the pharmaceutically acceptable salts or esters thereof, or stereoisomers thereof. Such compounds can be represented by Formula (I):

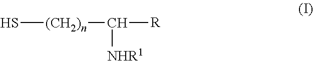

the pharmaceutically acceptable salts or esters thereof, and stereoisomers thereof, wherein:
R═H, CONHCH₂COOH, NH₂ or COOR² wherein R² is H or C₁₋₄alkyl;
R¹═H, COCH₃, CONH₂, or CO(CH₂)ₘCH(NH₂)(COOH) wherein m is 1 or 2; and
n=a number having a value of from 1 to 4.

Illustrative examples of compounds of Formula (I) include those shown in the following list:

### List of Non-limiting Examples of Sulfhydryl Compounds

Cysteine (l-cysteine, d-cysteine, dl-cysteine)

### N-Acetyl-l-cysteine

dl-Homocysteine
l-Cysteine methyl ester (methyl cysteine)
l-Cysteine ethyl ester (ethyl cysteine)
N-Carbamoyl cysteine

### Glutathione

### Cysteamine

The preferred compounds for use in the invention are cysteine, glutathion and N-acetyl-l-cysteine.

Method of Coating a Sulfhydryl Compound onto Galvanic Particulates

A nonlimiting exemplary procedure to prepare the sulfhydryl compound coated galvanic particulates according to the present invention is following:
(a) Prepare a solution of a sulfhydryl compound of certain concentration in a polar solvent or a mixture polar solvents
(b) With mixing, add certain amount galvanic particulates into the sulfhydryl compound solution
(c) Allow enough time for the coating reaction to complete
(d) Separate the coated galvanic particulates from the solution by a filtration process
(e) Dry the coated galvanic particulates

Polar solvents of the invention include, but are not limited to water, ethyl alcohol, propylene glycol, butylenes glycol, glycerin, polyethylene glycol. The suitable concentration of the sulfhydryl compound in the solution for the coating process ranges from about 0.1% to about 90% or to the solubility of a given solute and solvent composition. The amount of galvanic particulates to be used for the coating process is determined by the reaction equipment, namely, the ability to achieve through mixing and the quantity of the sulfhydryl compound used for a given reaction. The coating reaction time may vary from several minutes to several hours at ambient temperature depending on a coating rate of a given reaction. Elevated temperature may be used to accelerate a slow coating reaction if needed.

### Utilities of the Sulfhydryl Compound Coating on the Galvanic Particulates

In one embodiment, the sulfhydryl-containing amino acids or their derivatives and analogs thereof are employed in amounts sufficient to coat, either partially or completely, on the surface of a galvanic particulate in the controlled manner to regulate generation of galvanic electricity and to improve the chemical stability of the galvanic particulate. The coating also serves an intermediate layer to promote compatibility of the galvanic particulate with the biological cells and tissues, especially when a sulfhydryl-containing amino acid and its derivatives (e.g., L-cysteine, glutathion and N-acetyl-L-cysteine) are used to form the coating. Another important utility of such a coating is to enable attachment of other chemical and biochemical moieties to the galvanic particulate surface by chemical bonds such as a covalent bond (e.g., an ester bond on the carboxyl group of an amino-acid or amino-acid derivative) with the sulfhydryl compound for intended biological effects. One non-limiting example of utilizing such an attachment method is to attach another compound (e.g, a drug or an active agent) via covalent bonding to the sulfhydryl compound immobilized on the surface of a galvanic particulate to form a pro-drug affixed at the surface. Upon application to human body (e.g., via oral, injectable, implantable or surgical route), the esterase will cleave the ester bond to release the drug at the close vicinity of the galvanic particulate to exert its own pharmacological activity together with the biological activities of the galvanic particulates for a synergized action. The bonding site of another compound (i.e., the second compound) to the sulfhydryl compound can be at many functional groups of the second compound, and can be any type of the bond (i.e., not limiting only to ester bond), as long as the bond can be cleaved at the application site in the body environment. The pro-drug approach is well known in the art of pro-drug design and synthesis in medicinal chemistry. In another embodiment, the second compound can be a diagnostic agent or marker, instead of a drug or active agent, to be used diagnostic purpose.

### Galvanic Particulate Flakes as Cosmetic Metallic

### Effect Pigment

In one embodiment of the invention, aforementioned galvanic particulates are in the form of metallic effect pigment to provide both biological activities as well as the attractive appearance such as the metallic materials used in color cosmetic products. Metallic effect pigments are used widely in cosmetic compositions, which are usually manufactured with base metallic flakes often chosen from metals such as aluminum, copper, or copper-zinc alloys. The term metallic effect pigments is used to denote metallic pigments which have a directed reflection at oriented, metallic or highly light-refractive particles of a predominantly flat configuration. They are always of plate-like or flake-like configuration and have very large particle diameters compared with dye pigments. Their optical properties are determined by reflection and interference. Depending on transparency, absorption, thickness, single layer or multi-layer structure, the special-effect pigments exhibit a metallic shine, a pearl shine, interference or interference reflection. The silvery color of metallic aluminum flake can be altered to many colors by a partial coating to the base metal surface with fine particles of a metal oxide such as iron oxide, copper oxide, their mixture, or other metal oxides using a binder systems such as an organic polymer, a silicate or silica. The partial coating on the metallic effect pigment provides (a) attractive color or esthetic metallic shine, a method of controlling generation of galvanic electricity to reduce the intensity and thereby increasing its duration, and (c) a protection from oxidation for the base metal flake to maintain its metallic shine. Manufacturing methods of colored cosmetic effect pigments are disclosed in U.S. Pat. No. 5,931,996 and are hereby incorporated as reference in its entirety.

One embodiment of the present invention discloses a composition and method to use a galvanic particulate with the cosmetic optical properties of metallic effect pigment, in the form of flake shaped galvanic particulate with or without an incomplete colored or non-colored coating, for beneficial biological effects to the body of an animal such as human. Such galvanic metallic effect pigment is particularly useful for cosmetic applications which simultaneously provide the user only the benefits of electric stimulation and electrochemically mediated changes from the galvanic particulates, but also the attractive esthetic appearance of the optical properties of a metallic effect pigment.

Advantages of using galvanic metallic effect pigments for topical application of human barrier membranes and adjacent tissues (skin, mucosa, hair, wound, lips, teeth, etc.) include, but are not limited to, (a) high activity of beneficial galvanic action due to the high specific surface area; (b) attractive appearance of metallic effect pigment with a wide range of color options; (c) ability to change from a metallic shinning appearance to a less shinning appearance after being applied to the moist barrier membrane such as the skin to blend gradually into a natural look of a healthy skin.

Preferred manufacturing method for coating the second conductive metal to the first conductive metal flake to form the galvanic metallic effect pigments of the present invention is by a vapor deposition, either a physical vapor deposition or chemical vapor deposition well known in the art in metallic effect pigment industry. Manufacturing methods of colored cosmetic effect pigments are disclosed in U.S. Pat. Nos. 5,931,996, 5,964,936, 5,993,526 and 7,172,812, and are hereby incorporated as reference in their entirety.

Absorbable filler Composition Comprising Galvanic Particulates

One embodiment of the present invention discloses a method of enhancing the performance of cosmetic injectable fillers, such as collagen filler injection for wrinkles by (a) stimulating endogenous collagen and elastin synthesis for prolonged efficacy; (b) reducing complications associated with the filler injection procedure, such as undesirable inflammatory tissue responses to the injection (e.g., pain, tenderness, edema, and skin erythema), and potential infection.

According to a publication by TK Hamilton, MD (Skin Aurgumetnation and correction: the new generation of dermal fillers—a dermatologist's experience, Clinics in Dermatology, 2009:27, S13-S22), cosmetic injectable dermal fillers for moderate to deep facial wrinkles and folds can be categorized into two groups: (a) replacement dermal fillers, such as bovine and human collagens and hyaluronic acid, that restore soft tissue volume loss by fill in the deep dermis and subcutaneous space (duration: 3-12 months); and (b) stimulatory fillers, such as Poly-L-lactic Acid (PLLA) and calcium hydroxylapatite (CaHA), that replace volume by stimulating fibroblast activity, collagen synthesis and soft tissue growth (duration: 9-24 months). Commercial collagen and hyaluronic acid fillers in the U.S. (e.g., Evolence by Ortho Dematologics, and Zyderm, Zyplast, CosmoDerm& CosmoPlast by Allergan) are formulated as viscous gel injection compositions, and the commercial injectable PLLA filler in the U.S. (Sulptra by Dermik) are formulated as a lyophilized powder that requires reconstitution with sterile water for injection. The drawback of replacement dermal fillers is their short duration and required frequent treatment, whereas the drawback for stimulatory fillers is side effects. For example, adverse event associated with injectable PLLA include bruising, edema and inflammation. In addition, there is pain associated with filler injections and lidocaine are commonly used in the injection composition for pain control.

One embodiment is to overcome these drawbacks by co-administering galvanic particulates of this invention by mixing them with the filler composition prior to the injection. Because biological effects of the galvanic particulates include anti-inflammation, stimulation of collagen and elastin synthesis, antimicrobial activity and pain reduction (see Examples), addition of galvanic particulates to a replacement filler injection can prolong the duration, and to a stimulatory filler injection can reduce side effects such as inflammation, edema, bruising and pain. Preferred galvanic particulates for the filler application are those made of metals that can be turned to essential minerals for the human body, such as zinc and magnesium as anode material (the first conductive metal) and copper and iron (the second conductive metal) such as galvanic particulates of Zn—Cu, Mg—Cu, Mg—Fe. The same preference holds true for the following oral application described in the next section.

### Peroral Treatment for Obesity and Weight Control Using an Galvanic Particulate Composition

Obesity is a global problem with estimated 300 million people worldwide. It is particular serious epidemic for the U.S. population that affects approximately 60 million people in the U.S. Women are especially affected. Over one-third of women between the ages of 20 and 74 are obese. Even more people are over-weight now and are progressively approaching the clinical definition of obesity. Electric stimulation has been proposed to treat obesity. A publication by Y. Sun and J. Chen has demonstrated electric stimulation's effect to reduce fat absorption (“Intestinal electrical stimulation decreases fat absorption in rats: therapeutic potential for obesity”, Obesity Research, Vol. 12, No. 8, August 2004, pages 1235-1242). U.S. Pat. No. 7,177,693 disclosed an implantable gastric electric stimulation device to treat obesity. Given the huge costs and potentially serious complications associated with surgical implant electric stimulation devices, as well as the epidemic scale of the population involved with obesity and weight problem, a non-surgical approach is clearly the preferred treatment method.

One embodiment of the present invention discloses a method to provide electric stimulation to the gastrointestinal (GI) tract of an animal such as human using an oral galvanic particulate composition for weigh control and for treating obesity. The oral galvanic particulate composition comprising galvanic particulates made of metallic components that will turn into pharmaceutically and nutritionally acceptable mineral ions after the galvanic actions, and a controlled release oral dosage form which protects galvanic particulates from premature degradation and allows galvanic particulates activated at the target site in the gastrointestinal tract (e.g., the small intestine to reduce fat absorption or certain target site such as jejunum).

Advantages of using galvanic particulates for gastrointestinal electric stimulation include, but are not limited to, (a) provide electric stimulation to a target site in GI tract without the need of surgically implanted electric stimulation device (e.g., aforementioned gastric pacing device and intestinal pacing device) to eliminate any surgery associated risks, cost and post-surgical complications; (b) good safety because electric stimulation action is confined at the target site within gastrointestinal tract, and the by-products of the galvanic electric stimulation are essential minerals already in daily diet and nutritional supplements; (c) significant cost reduction in comparison to the surgical implant option; (d) convenient applications and easy treat termination if needed.

One embodiment of this invention is to formulate galvanic particulates into an oral dosage form typically for oral drug administration, such as a hard gelatin capsule, soft gelatin capsule and tablet, well known in the art in pharmaceutical industry and oral pharmaceutical products. Such an oral dosage form containing the galvanic particulates upon ingestion can deliver the galvanic particulates to the GI tract to provide galvanic electric stimulation. Depending on the target stimulation site, the oral dosage form can be designed for fast release (e.g., as fast release or rapid disintegrating table) for gastric electric stimulation, or can be coated with, or embedded within enteric polymer(s) (e.g., Eudragit S, Eugragit R polymers) which are insoluble and water-impermeable in the acidic gastric fluid to protect the galvanic particulates keep them in a non-activated state, but readily dissolves when approaching neutral pH in the small intestine to release the galvanic particulate for electric stimulation action.

A recent publication has explored the potential relationship between the types of microbiota found the intestine and the regulation of the body weight because the intestinal bacteria in obese humans and mice differ from those in lean individuals (“Obesity and gut flora” by M. Bajzer and R. J. Seeley, Nature, Vol. 444, 21/28 Dec. 2008, page 1009-1010). It has been speculated the type of bacteria in obese individual are more efficient in breaking down the food thus helping the host to absorb more calories. Because the galvanic particulates of this invention have demonstrated antimicrobial activity and can be used to control the bacterial quantity in the intestine, therefore can help to reduce intake of calories in addition to provide intestinal electric stimulation. One embodiment of the invention is to reduce intestinal microbial content by per oral administration of a pharmaceutically or nutritionally acceptable oral dosage form product or composition to treat or prevent obesity or overweight.

The present invention is illustrated below the following non-limiting Examples.

### Example 1

### Galvanic Particulate Preparation Based on Displacement Chemistry (Coating-Formation Method)

0.1% copper coated zinc galvanic particulates were manufactured by electroless plating of copper onto zinc powder. 10 g of ≦45-micron zinc powder was spread evenly onto a vacuum filter buchner funnel with a 0.22 micron filter. 5 g of copper acetate solution was then poured evenly onto the zinc powder, and allowed to react for approximately 30 seconds. A suction was then applied to the filter until the filtrate was completely suctioned out. The resulting powder cake was then loosed, and 10 g of deionized water was added and then suctioned off 10 g of ethanol was then added to the powder under suction. The powder was then carefully removed from the filter system and allowed to dry in a desiccator.

### Example 2

### Elastin and Collagen Gene Expression in Human Skin Explant Model

Skin ageing is associated with decreased collagen and elastin synthesis and increased degradation of collagen and elastin fibers. Thus, agents able to enhance their synthesis or protect it from degradation, could be beneficial for prevention or reduction of skin ageing. Products that are able to enchance elastogenesis and collagen synthesis has the potential to deliver numerous consumer benefits not only for skin anti-ageing but also in the area of Women's Health (FSE, incontinence, prolapse etc.), wound healing, improve oral health and reduce or prevent the occurrence of hemorrhoids to name a few.

Surprisingly, we found that Zn—Cu galvanic particulate prototypes were able to effectively promote elastin and collagen expression in vitro in a human skin explant model. An increase in the number and thickness of elastin fibers and the presence of newly formed collagen in the dermis of swine skin was also histologically observed after treatment with Zn—Cu galvanic particulate prototypes. This increase is associated with improvement of skin structure, elasticity and resilience, and can be used to prevent and correct skin ageing.

The skin explant model was established using human skins obtained with informed consent from abdominal skins of healthy individuals undergoing plastic surgery. Patient identities were not disclosed to preserve confidentiality, in compliance with US HIPAA regulations. Punch biopsies (12 mm) were maintained in DMEM/F12 base medium, supplemented with a cocktail of growth factors.

Skin explants were cultured in culture media for 1 day followed by once daily topical treatment of 15 μl of a freshly prepared 1% suspension in water of 0.1% Cu coated Zn galvanic particulates made according to Example 1. Skin samples were collected at 5 and 7 days in culture. Harvested samples were either processed for histology H&E, Luna elastin staining, or RNA analysis for elastin and collagen IV mRNA levels. Images of histology stained sections were obtained by ImagePro Plus 5.1 (Mediacybernetics, Silver Spring, Md.). Real-time PCR were performed with the extracted RNA.

The results are shown in Table 1.

TABLE 1

| | | Elastin mRNA, | Collagen IV |
|---|---|---|---|
| | | real-time PCR | mRNA, |
| | Score of elastin fiber | (% change | real-time PCR |
| | production by | relative to | (% change |
| | visual assessment | control, at | relative to |
| Test | of images (Luna | day 5, 7, | control, at day |
| material | Staining, at day 7) | respectively) | 5, 7 respectively) |
| H₂O | 0 | 100% | 100% |
| (control) | (baseline) | | |
| 1% suspension | 2 | 1100%, 280% | 214%, 180% |
| of Zn-Cu | (in a scale from 0-3) | | |
| galvanic | | | |
| particulates | | | |
| in H₂O | | | |

The data in Table 1 demonstrates Zn—Cu galvanic particulates increased the amounts of elastin fibers, especially in the papillary dermis perpendicular to the DE junction and increased both elastin and collagen IV mRNA levels. In addition, H&E staining showed that no tissue damage or architectural changes were induced by Zn—Cu galvanic particulates (H&E staining).

### Example 3

### Enhancement of Collagen and Elastic Fiber Network in Swine Skin by Histological Analysis after the Treatment with Zn—Cu Galvanic Particulate Prototypes

A medium tanned Yucatan Micro swine was obtained from Charles River (Portland, Me.), housed in the appropriate sized single cage, in an environmentally controlled room with a 12-hour light-12-hour dark photoperiod and supplied with 600 g standard pig food per day and water ad libitum. Animal care was based on the “Guide for the Care and Use of Laboratory Animals”, NIH Publication No. 85-23. The animal was acclimated for a week before topical areas were demarcated by standard tattooeing procedures and allowed to heal an additional week prior to commencement of the study.

Topical agents (200 μl per site on each side) were applied twice a day for, 5 days a week for 8 weeks to tattooed sites (approximately 5 hours apart). The swine skin was wiped with a wet paper towel to remove excess dry skin and dried prior to treatment. The treatments contained 1% and 5% suspensions in water of 0.1% Cu coated Zn galvanic particulates made according to Example 1 and a water only treatment as control. Location of the treatment sites was randomized on both sides of each swine.

Eight weeks after the start of the topical treatments and 24 hours after the last treatment, the swine was humanely euthanised via an intravenous injection of a sodium pentobarbital based drug. Skin punch biopsies (8 mm) were collected from all treatment sites, fixed in 10% Neutral formalin. Paraffin sections of swine skin samples were analyzed histologically for new collagen production (Procollagen stain) and elastin fibers (Luna's elastin with trartrazine counterstain). Surprisingly, increased amounts of elastin fibers were observed in the papillary dermis perpendicular to the DE junction as well as the presence of thicker and increased fibers in the reticular dermis in swine skins treated with Zn—Cu galvanic particulate, as compared to controls. Procollagen staining confirmed that newly formed/young collagen (blue staining) was present throughout the dermis in skins treated with Zn—Cu galvanic particulate compared to controls containing mature collagen (red staining).

### Example 4

### Anti-Inflammatory Activity on Release of UV-Induced Pro-Inflammatory Mediators on Reconstituted Epidermis

Following examples demonstrated galvanic particulate's anti-inflammatory activity in vitro when mixed with a commercial collagen fill injection for wrinkle treatment.

The effect of a collagen-based dermal filler containing galvanic particulate was evaluated for anti-inflammatory activity on human epidermal equivalents. Epidermal equivalents (EPI 200 HCF), multilayer and differentiated epidermis consisting of normal human epidermal keratinocytes, were purchased from MatTek (Ashland, Mass.). Upon receipt, epidermal equivalents were incubated for 24 hours at 37° C. in maintenance medium without hydrocortisone. Galvanic particulates (0.1% Cu-coated Zn galvanic particulates made according to Example 1) were mixed with a collagen-based dermal filler (Evolence® commercially available from OrthoDermatologics) to produce a dermal filler containing 1% of the galvanic particulates. The collagen-based dermal filler containing 1% galvanic particulates or the collagen-based dermal filler alone were applied to the skin equivalents respectively for 2 hours before exposure to solar ultraviolet light (1000W-Oriel solar simulator equipped with a 1-mm Schott WG 320 filter; UV dose applied: 70 kJ/m2 as measured at 360 nm). Equivalents were incubated for 24 hours at 37° C. with maintenance medium then supernatants were analyzed for IL-1a cytokine release using commercially available kits (Upstate Biotechnology, Charlottesville, Va.).

The results are shown in Table 2.

TABLE 2

| Treatment | Mean +/− Std Dev of IL-1A | Percent Inhibition of |
|---|---|---|
| (Dose, as % w/v) | Release (ng/ml) | Skin Inflammation |
| Untreated, No UV | 1.18 ± 0.18 | — |
| UV (60 KJ), collagen- | 176.07 ± 351.36 | — |
| based dermal filler | | |
| UV (60 KJ) + collagen- | 661.74 ± 135.78** | 44% |
| based dermal filler | | |
| containing 1% galvanic | | |
| particulates | | |

**Indicates significant difference from UV + collagen-based dermal filler alone, using a student's t-Test with significance set at P < 0.05.

The collagen-based dermal filler containing 1% galvanic particulates was able to significantly reduce the UV-stimulated release of inflammatory mediators. Therefore, collagen-based dermal filler containing galvanic particulates would be expected to provide an effective the anti-inflammatory benefit.

### Example 5

### Oral Administration of Galvanic Particulates Produced a Analgesic Effect

Pain is defined as “an unpleasant sensory and emotional experience associated with actual or potential tissue damage, or described in terms of such damage”—International Association for the Study of Pain (IASP). Pain can range from unpleasant to debilitating. Pain can occur during disease states such as arthritis or can occur as a result of surgical procedures, such as after injections or incisions, or subsequent to laser treatments. Analgesic agents can reduce pain, however there are many side effects associated with their use. For example, aspirin can cause gastrointestinal damaged, and opioid compounds can induce conditions ranging from addiction to decreased respiratory function. Therefore the need exists for novel analgesic agents to treat pain.

In this example, galvanic particulates produced by the process of Example 1 were evaluated for analgesic activity. Aspirin, a non-steroidal analgesic agent, which is known to show efficacy in this model, was included as a reference compound. Albino male CD-1 mice, weighing 24±2 g were used in the study. Galvanic particulates or aspirin were prepared in deionized water (DI Water) and were administered orally at a concentration of 100 mg/kg to mice 1 hour prior to injection of acetic acid. A 20 mg/kg of a 0.5% acetic acid solution was injected intraperitoneally, and the number of acetic acid-induced writhes were counted by an observer blinded to the treatment groups. A 50% decrease in the number of writhes indicated analgesic activity.

TABLE 3

| Treatment | Total Number of Writhes | Analgesic Effectiveness |
|---|---|---|
| (Dose) | (Mean ± Std Dev) | (Percent Reduction Writhes) |
| Vehicle | 11.4 ± 6.1 | — |
| (DI Water) | | |
| Galvanic | 4.6 ± 4.3* | 60% |
| particulate | | |
| Aspirin | 5.0 ± 3.6* | 56% |

** = P < 0.05 Compared to Vehicle using a Student's t-Test

The galvanic particulates produced an analgesic activity in a pain model comparable to an analgesic drug.

### Example 6

### Demonstration of Galvanic Particulate's Antimicrobial Activity Using In Vitro Tests

### Test Materials:

Powder Samples:
copper coated zinc galvanic particulates made according to the process of Example 1:
   0.1% Copper (Cu) coated Zinc (Zn)
   0.01% Cu coated Zn
Zn powder
Cu powder

### Test Microorganisms:

Aspergillus niger American Type Culture Collection (ATCC) 16404 Quanti-Cult Plus, Chrisope Technologies, A Division of Remel Inc., Product #47-11100
Campylobacter jejuni subsp. jejuni ATCC 33291, Culti-loops, Remel Inc., Product #R4601400
Candida albicans ATCC 10231, Quanti-Cult Plus, Chrisope Technologies, A Division of Remel Inc., Product #47-11503
Clostridium difficile ATCC 43594, freeze-dried isolate, ATCC
Enterococcus faecium ATCC 700021 (vancomycin resistant [VRE]), supplied by Ethicon Microbiology Group
Escherichia coli ATCC 8739, Quanti-Cult Plus, Chrisope Technologies, A Division of Remel Inc., Product #47-17085
Haemophilus influenzae ATCC 49247, Culti-loops, Remel Inc., Product #R 4603830
Listeria monocytogenes ATCC 7644, Culti-loops, Remel Inc., Product #R4603970
Moraxella catarrhalis ATCC 8176, Culti-loops, Remel Inc., Product #R4601228
Propionibacterium acnes ATCC 6919, Culti-loops, Remel Inc., Product #R4607101
Pseudomonas aeruginosa ATCC 27853, Culti-loops, Remel Inc., Product #R4607060
Salmonella enterica serovar Typhimurium ATCC 14028, Culti-loops, Remel Inc., Product #R4606000
Staphylococcus aureus ATCC 33593 (methicillin resistant [MRSA]), supplied by Ethicon Microbiology Group
Streptococcus pneumoniae ATCC 49619, Culti-loops, Remel Inc., Product #R4609015
   Streptococcus pyogenes ATCC 19615 Group A, Culti-loops, Remel Inc., Product #R4607000
Trichophyton mentagrophytes ATCC 9533, Culti-loops, Remel Inc., Product #R4608300
BacT/ALERT iAST—40 mL supplemented tryptic soy broth (TSB), standard aerobic bottle, Product #259786
BacT/ALERT iNST—40 mL supplemented TSB, standard anaerobic bottle, Product #259785
BacT/ALERT MB—29 mL supplemented Middlebrook 7H9 broth, Product #251011, with 1-mL MB/BacT enrichment fluid, Product #259877, for Mycobacteria cultivation from blood samples.

### Test Equipment

BacT/ALERT Microbial Detection System, Model 240, Serial #001BT2893
Biological safety cabinet
Incubator set at 33° C.

### Test Microorganism Culture Preparation

Quanti-Cult Plus samples were suspended according to manufacturer's instructions and injected into appropriate BacT/ALERT sample bottles. Most Culti-Loop samples were aseptically clipped into individual 1.5-mL sterile polypropylene screw-capped tubes and then dissolved in approximately 1 mL of sterile TSB. The 1-mL sample aliquots were injected into appropriate BacT/ALERT sample bottles. The sample bottles were then incubated in the BacT/ALERT system to obtain stationary phase cultures. Stationary phase cultures of MRSA and VRE were kindly supplied by the Ethicon Microbiology Group. The BacT/ALERT incubation temperature varied from 33-37° C., depending on the optimum growth requirements of the test microorganisms.

To culture T. mentagrophytes a Culti-Loop was streaked directly onto the surface of a TSA plate and incubated for 3-7 days at 33° C. to generate mycelial growth on the plate surface.

Aerobic BacT/ALERT iAST bottles were used to culture most of the test microorganisms, with the exceptions of P. acnes, C. difficile, C. jejuni and H. influenzae, where anaerobic BacT/ALERT iNST bottle were utilized. To optimize the growth of C. jejuni and H. influenzae the anaerobic media bottles were supplemented with 5-mL of defibrinated sheep blood and approximately 8-mL of sterile air was aseptically injected into the bottle headspace to produce microaerophilic growth conditions.

### Zn—Cu Galvanic Particulate, Zn Powder and Cu Powder Suspensions

Exactly 0.4 g of the galvanic particulate samples was weighed out into individual 1.5-mL sterile screw-capped polypropylene tubes. Approximately 1-mL media aliquots from designated BacT/ALERT sample bottles were then pulled up into a 3-mL sterile syringe using a sterile 20 G needle. This aliquot was used to resuspend the powdered sample and then injected back into the 40-mL sample bottle to obtain a 1% galvanic particulate suspension. This procedure was repeated until the galvanic particulate was quantitatively transferred aseptically into the BacT/ALERT sample bottles. The same procedure was also used to transfer 0.4 g of zinc powder and 0.04 g of copper powder to obtain final suspensions of 1% and 0.1% respectively, to serve as process controls.

### Test Microorganism Suspensions

A 1-mL sterile syringe and 20 G needle was used to inject 0.5-mL aliquots of stationary phase test microorganism cultures into designated BacT/ALERT sample bottles containing 1% galvanic particulates, 1% Zn powder, or 0.1% Cu powder, to obtain a target population concentration of approximately 1×10⁶CFU/mL. Actual delivery counts were checked by dilution pour plating using molten TSA and are shown in Table 4 below. For P. acnes and C. albicans an additional population concentration of 1×10²CFU/mL was also tested to investigate whether the antimicrobial efficacy of galvanic particulates was concentration dependent for these test microorganisms.

In the case of T. mentagrophytes an approximate 1 cm² agar plug was aseptically excised from the TSA plate containing the surface mycelia and transferred into a 250-mL sterile shake flask containing glass beads and 50-mL of sterile DI water. The shake flask was vortexed until a turbid white suspension was obtained containing predominantly fragmented mycelia. A 1-mL sterile syringe and 20 C needle was used to inject 1-mL of this turbid suspension into designated BacT/ALERT sample bottles containing 1% galvanic particulates, 1% Zn powder, or 0.1% Cu powder. As mentioned above the actual delivery count was determined and is shown in Table 4 below. For this test microorganism an additional minimal media aerobic BacT/ALERT sample bottle (BacT/ALERT MB) was included to look into possible media effects on galvanic particulates antimicrobial efficacy.

### BACT/ALERT Antimicrobial Analysis

The designated BacT/ALERT samples bottles containing the test microorganism and powder suspensions were loaded into the BacT/ALERT system where they were continuously agitated and automatically monitored for growth. The BacT/ALERT incubation temperature varied from 33-37° C., depending on the optimum growth requirements of the test microorganisms. The incubation time was set for 7-days, at which time, if no growth was detected the sample was flagged as negative for growth. Appropriate positive and negative process controls were included for each sample set.

Negative sample bottles were then subcultured by injecting a 1-mL aliquot into a new BacT/ALERT sample bottle, to help determine the galvanic particulates' bactericidal versus bacteriostatic activity, as shown in Table 4 below. The galvanic particulates were determined to be bacteriostatic against a designated test microorganism when microbial outgrowth was detected following an additional 7-day incubation of the 1-mL subcultured sample bottle. However, since no microorganism identification was performed on these positive subculture samples, the possibility of having introduced a contaminant during the subculturing process cannot be conclusively ruled out in this preliminary study. In addition, in the cases of P. aeruginosa and E. coli, the bacteriostatic results may be a result of the higher starting test microorganism cell concentration of 1×10⁷ vs. 1×10⁶ CFU/mL, which were inadvertently delivered into the BacT/ALERT sample bottles.

### Results and Discussion

The antimicrobial activity of 1% galvanic particulates suspended in TSB-based BacT/ALERT media sample bottles inoculated with representative target-specific pathogenic test microorganisms are presented below (Table 4). The discussions are organized by potential medical indications. The 0.1% Cu powder only controls did not inhibit the growth of the test microorganisms. Where indicated by an asterisk (*) the 1% Zn powder control was also found to be bacteriostatic or bactericidal to the test microorganisms. In addition to antimicrobial efficacy and proven anti-inflammatory, wound healing and analgesic properties, the copper/zinc galvanic particulates' associated electrical current may inhibit pathogenic microorganism quorum sensing (QS), which is crucial for setting up biofilms and pathogenic infections, thereby enhancing the galvanic particulate treatment efficacy. Thus in cases where the galvanic particulates were found to be bacteriostatic or even non-inhibitory to growth, in the case of C. difficile for the treatment of Colitis as shown in Table 4 below, treatment efficacy may still be present due to the inhibition of biofilm/infectivity. In addition, the possible antimicrobial efficacy of galvanic particulates against lower population concentrations of C. difficile (i.e., 1×10² CFU/mL) for the possible prevention/treatment of Colitis cannot be ruled out without further studies.

It should be noted that the test concentration of the galvanic particulates was arbitrarily chosen as 1%, and the copper coating level range of 0.01%-0.1% for test purpose to produce these antimicrobial results. Additional in vitro studies were performed (results not shown) that demonstrated the antimicrobial efficacy of the 0.1% copper coated zinc galvanic particulates down to a 0.1% test concentration, where the Zn powder alone did not show inhibition to growth. To increase the antimicrobial activity of Zn—Cu galvanic particulates, one can increase the galvanic electricity generation by modifying three factors, separately or simultaneously: (1) increasing galvanic particulate concentration; (2) increasing copper content to increase the galvanic reaction area; and (3) increasing zinc specific surface area such as by reducing the particle size of the elemental zinc particles.

Current literature supports the use of a copper/zinc galvanic particulate application as an antibiotic adjunct to increase treatment efficacy and inhibit the formation of antibiotic resistant microorganisms. A recently published article shows that the three major classes of antibiotics, regardless of drug-target interaction, stimulate the production of highly deleterious hydroxyl radicals in Gram-negative and Gram-positive bacteria, which ultimately contribute to cell death. This suggests that the reactive oxygen species (ROS) produced by the galvanic particulates may serve as an adjunct to standard antibiotic therapy to increase treatment efficacy and reduce the formation of antibiotic resistant microorganisms. The possibility just mentioned, of the galvanic particulates' electric current interfering with QS to inhibit biofilm formation, would also serve as an added benefit for using galvanic particulates as an antibiotic adjunct.

TABLE 4
Zn-Cu galvanic particulate BacT/ALERT Antimicrobial Results:

| | | Approximate | |
|---|---|---|---|
| | Test | Concentration | |
| Medical Indication | Microorganism | CFU/mL | 0.1% Cu coated Zn^{#} |
| Acne | P. acnes | 1 × 10⁶ | Bacteriostatic* |
| | | 1 × 10² | Bactericidal* |
| Nosocomial | MRSA | 1 × 10⁶ | Bactericidal |
| Infection | VRE | 1 × 10⁶ | Bacteriostatic |
| Colitis | C. difficile | 1 × 10⁶ | Positive for Growth |
| Sinusitis | M. catarrhalis | 1 × 10⁶ | Bactericidal* |
| | H. influenzae | 1 × 10⁶ | Bactericidal* |
| Sinusitis | S. pneumoniae | 1 × 10⁶ | Bactericidal* |
| Otitis Media | | | |
| Otitis Externa | P. aeruginosa | 1 × 10⁷ | Bacteriostatic |
| Foodborne Ilness | C. jejuni | 1 × 10⁶ | Bactericidal* |
| | L. monocytogenes | 1 × 10⁶ | Bactericidal |
| | S. enterica | 1 × 10⁶ | Bactericidal |
| Foodborne Ilness | E. coli | 1 × 10⁷ | Bacteriostatic |
| Urinary Tract | | | |
| Infection | | | |
| Strep Throat | S. pyogenes | 1 × 10⁶ | Bactericidal* |
| Topical Yeast or | C. albicans | 1 × 10⁶ | Positive for Growth |
| Fungal Infections | | 1 × 10² | Bacteriostatic |
| | A. niger | 1 × 10⁶ | Bactericidal |
| | T. mentagrophytes | 1 × 10⁵ | Bactericidal |
| | | | (media dependent) |

^{#}0.01% Cu coated Zn; Bacteriostatic* for 1 × 10⁶ CFU/mL P. acnes, Bactericidal for MRSA, Positive for growth for VRE.

### Example 7

### Multiphase Galvanic Particulate Preparation from Molten Zinc and a Unmelted Copper Powder

Fine particles of elemental copper and elemental zinc were formed first by separate atomization of molten copper and molten zinc. At 1:1 weight ratio, the fine particles of copper and zinc were then spray-formed and atomized together at a process temperature above 420° C. to enable the molten zinc particles to sinter together with the copper particles. Aggregate composite particles of Zn and Cu were formed. The process was carried out under a protective argon gas atmosphere. The resulting multiphase Zn—Cu galvanic particulates had a Zn:Cu weight ratio of 1:1, and particle size less than 100 mesh (i.e., smaller than 149 microns).

An SEM image of the multiphase Zn—Cu galvanic particulates clearly showed that the surface of the galvanic particulates consisted of two distinct, randomly distributed, metallic domains (i.e., copper and zinc with their characteristic colors). A majority of the galvanic particulates were smaller than 100 microns. The result of surface analysis of the multiphase Zn—Cu galvanic particulates using Energy Dispersive X-Ray Spectroscopy (EDS) is shown Table 5. Sample 1 was Zn—Cu galvanic particulates made in accordance with Example 1.

TABLE 5

| Zn-Cu Galvanic | | % of Copper | % Surface |
|---|---|---|---|
| Particulate | Manufacturing | in Zinc | Coverage of |
| Sample No. | Method | (by weight) | Cu (by EDS ) |
| 1 | Example 1 | 5 | 19-28 |
| | (coating) | | |
| 2 | Example 7 | 50 | 21-30 |
| | (melting/dispersion) | | |

The surface coverage by copper was different between galvanic particulates made by coating (Example 1) and multiphase galvanic particulates made by melting/dispersion (Example 7). In general, a greater surface coverage can be achieved with smaller amount of first conductive metal (i.e., copper) using a coating method for making galvanic particulates compared with a melting/dispersion method for making galvanic particulates. However, a relatively more consistent galvanic action for electricity generation is expected for the multiphase galvanic particulates throughout their electricity-generating life, because of a more homogeneous first and second conductive material distribution therein. This is in contrast to the galvanic particulates made using coating methods, in which the second conductive material is only coated on the surface of the first conductive material.

### Example 8

### Topical Anti-Inflammatory Activity in a Murine Model of Contact Hypersensitivity

The ability of topically applied of multiphase Zinc-Copper galvanic particulates (made as described in Example 7) to affect the inflammatory response was demonstrated using an in vivo immune cell-mediated skin inflammation model in comparison to Zn—Cu Galvanic Particulates made according to Example 1.

Albino male CD-1 mice, 7-9 weeks old, were induced on the shaved abdomen with 50 μl of 3% oxazolone in acetone/corn oil (Day 0). On Day 5, a 20 μl volume of 2% oxazolone in acetone was applied to the dorsal left ear of the mouse. Multiphase Zn—Cu galvanic particulates were applied to the left ear (20 μl) 1 hour after oxazolone challenge in a 70% ethanol/30% propylene glycol vehicle. The right ear was not treated. The mice were sacrificed by CO₂ inhalation 24 hours after the oxazolone challenge, the left and right ears were removed and a 7-mm biopsy was taken from each ear and weighed. The difference in biopsy weights between the right and left ear was calculated. Anti-inflammatory effects of compounds are evident as an inhibition of the increase in ear weight. The following results were obtained:

TABLE 6

| | Percent Inhibition of |
|---|---|
| Treatment (Dose) | Skin Inflammation* |
| Hydrocortisone (1 mg/ml) | 70.3% ± 6.6% |
| Coated Zn-Cu Galvanic Particulates | 81.3% ± 6.2% |
| (1 mg/ml) (made according to Ex. 1) | |
| Multiphase Zn-Cu Galvanic Particulates | 72.5% ± 6.1% |
| (1 mg/ml) | |

*% Inhibition = (Vehicle treated biopsy weight − Agent(s) treated biopsy weight)/(Vehicle treated biopsy weight) × 100

Topical application of the Zn—Cu galvanic particulates and the multiphase Zn—Cu galvanic particulates both demonstrated anti-inflammatory activity in a model of skin inflammation comparable to a corticosteroid (hydrocortisone). Furthermore, both Zn—Cu galvanic particulates and multiphase Zn—Cu galvanic particulates produced a reduction of inflammation comparable to hydrocortisone.

### Example 9

### Efficacy of Galvanic Particulates Against E Coli Versus Metal Powder Mixtures

Antimicrobial activities of the galvanic particulates manufactured by the coating method of Example 1 and the melting/dispersion method of Example 7 were evaluated in vitro, in comparison with physical mixtures of fine elemental zinc and copper powders of three ratios (i.e., Zn:Cu of 1:1, 2:1, and 10:1). Other test materials and test condition included: Trypticase soy agar (TSA), Escherichia coli (E. coli strain ATCC 8739), and Incubation time=24 hours at 37° C.

A modified zone of inhibition test was performed as follows. 0.1 g of metal powder (galvanic particulates or elemental zinc powder, or elemental copper powder) was dispended in 2 ml of deionized water with mixing, and then added to 8 ml melted TSA (final concentration of metal particles=1%). They were mixed, poured into a Petri plate and solidified. Discs (15 mm diameter) were punched from the agar and placed onto a lawn of bacteria on a TSA agar plate. The zones of inhibition were then measured following 24 hour incubation time. The results are shown in Table 7.

Both the coating method galvanic particulates and multiphase galvanic particulates demonstrated good and comparable antimicrobial activities against E. Coli under the test conditions. The mixtures of zinc powder and copper powder at ratio 1:1 showed significantly weaker antimicrobial activity than multiphase galvanic particulates, although it has an identical metal composition. As the Zn:Cu ratio of the metal mixtures was further increased to 2:1, even weaker antimicrobial activity was observed. When the Zn:Cu ratio was 10:1, no antimicrobial activity could be detected. These test results demonstrate the importance of galvanic action for the observed antimicrobial activity. Namely, the galvanic particulates, as preformed galvanic couples, had better galvanic antimicrobial action than the metal mixture of zinc and copper powders.

TABLE 7

| Material | Activity vs | Zone of |
|---|---|---|
| (1% total metal) | E. Coli | Inhibition (mm) |
| Ex. 1 Galvanic | Yes | 2.7 |
| Particulate | | |
| Zn:Cu = 1000:1 | | |
| Ex. 7 Galvanic | Yes | 2.6 |
| Particulate | | |
| Zn:Cu = 1:1 | | |
| Zn & Cu Powder | Yes | 1.2 |
| Mixture | | |
| Zn:Cu = 1:1 | | |
| Zn & Cu Powder | Yes | 0.7 |
| Mixture | | |
| Zn:Cu = 2:1 | | |
| Zn & Cu Powder | None | 0.0 |
| Mixture | | |
| Zn:Cu = 10:1 | | |

1. A multiphase galvanic particulate comprising a dispersed phase comprising a second conductive material dispersed in a continuous phase comprising a first conductive material, wherein both said first conductive material and said second conductive material are exposed on the surface of said particulate, the particle size of said particulate is from about 1 to about 500 microns, and said particulate comprises about 0.5 to about 60 weight percent of said dispersed phase.

2. The multiphase galvanic particulate of claim 1, wherein said dispersed phase has a melting point greater than about 950° C.

3. The multiphase galvanic particulate of claim 1, wherein said second conductive material is selected from the group consisting of copper, silver, gold, manganese, iron and alloys thereof.

4. The multiphase galvanic particulate of claim 1, wherein said continuous phase has a melting point of less than about 750° C.

5. The multiphase galvanic particulate of claim 1, wherein said first conductive material is selected from the group consisting of zinc, magnesium, aluminum, and alloys thereof.

6. The multiphase galvanic particulate of claim 1 further comprising a conductive/resistive interlayer.

7. The multiphase galvanic particulate of claim 6, wherein said conductive/resistive interlayer comprises an oxide or halide of said first conductive material or said second conductive material.

8. The multiphase galvanic particulate of claim 1, further comprising at least one additional dispersed phase comprising an additional conductive material.

9. The multiphase galvanic particulate of claim 1, wherein the difference in Standard Potentials of said first conductive material and said second conductive material is at least about 0.2V.

10. A method of treating the human tissue, which comprises applying to said tissue a multiphase galvanic particulate comprising a dispersed phase comprising a second conductive material dispersed in a continuous phase comprising a first conductive material, wherein both said first conductive material and said second conductive material are exposed on the surface of said particulate, the particle size of said particulate is from about 1 to about 500 microns, and said particulate comprises about 0.5 to about 60 weight percent of said dispersed phase.

11. The method of claim 10, wherein said human tissue is treated for inflammation.

12. The method of claim 10, wherein said human tissue is treated for infections and microorganisms.

13. The method of claim 10, wherein said human tissue is treated for regeneration.

14. The method of claim 10, wherein said human tissue is treated for wrinkles.

15. The method of claim 10, wherein said human tissue is treated for wound healing.

16. The method of claim 10, wherein said human tissue is treated for acne.

17. The method of claim 10, wherein said human tissue is treated for dermatitis.

18. An oral dosage form comprising the multiphase galvanic particulate of claim 1.
